(19) United States
(12) Patent Application Publication
(10) Pub. No.: US 2023/0326022 A1
(43) Pub. Date: Oct. 12, 2023

(54) Machine Learning Identification, Classification, and Quantification of Tertiary Lymphoid Structures

(71) Applicants: Bristol-Myers Squibb Company, Princeton, NJ (US); PathAI, Inc., Boston, MA (US)

(72) Inventors: Vanessa Matos-Cruz, Newton, PA (US); George Lee, North Wales, PA (US); Varsha Chinnaobireddy, Brookline, MA (US); Maryam Pouryahya, Bethesda, MD (US); Darren Thomas Fahy, Norwood, MA (US); Christian Winskell Kirkup, Somerville, MA (US); Kathleen Sucipto, Brookline, MA (US); Sai Chowdary Gullapally, Arlington, TX (US); Archit Khosla, New York, NY (US); Nishant Agrawal, Boston, MA (US); Benjamin Patrick Glass, Boston, MA (US); Sergine Brutus, Troy, MI (US); Limin Yu, Troy, MI (US); Murray Berle Resnick, Newton, MA (US); Rachel L. Sargent, Hillsborough, NJ (US); Vipul Atulkumar Baxi, Hillsborough, NJ (US); Scott Ely, Princeton, NJ (US); Benjamin J. Chen, Cambridge, MA (US)

(73) Assignees: Bristol-Myers Squibb Company, Princeton, NJ (US); PathAI, Inc., Boston, MA (US)

(21) Appl. No.: 18/297,281

(22) Filed: Apr. 7, 2023

Related U.S. Application Data
(60) Provisional application No. 63/422,763, filed on Nov. 4, 2022.
(60) Provisional application No. 63/329,352, filed on Apr. 8, 2022.

(51) Int. Cl. G06T 7/00; G06V 20/69; G06V 10/774; G06V 10/82; G16H 30/40; G16H 50/20; G16H 20/10

(52) U.S. Cl. CPC G06T 7/0012; G06V 20/698; G06V 10/774; G06V 10/82; G06V 20/695; G16H 30/40; G16H 50/20; G16H 20/10; G06T 2207/20036; G06T 2207/20081; G06T 2207/20084; G06V 2201/03; G06T 2207/30096

(57) ABSTRACT

A method includes receiving an input histology image, processing, using a cell classification model, the input histology image to generate one or more lymphocyte density maps within the input histology image, and performing morphological image processing on the one or more lymphocyte density maps to identify one or more TLS regions within the input histology image. Each TLS region is represented by a respective cluster of lymphocyte cells. For each corresponding TLS region of the one or more TLS regions identified in the input histology image, the method also includes extracting, from the respective cluster of lymphocyte cells, a respective set of TLS features, and processing, using a TLS classification model, the respective set of TLS features to classify the corresponding TLS region as one of a first TLS maturation state, a second TLS maturation state, or a third TLS maturation state.

### CROSS REFERENCE TO RELATED APPLICATIONS

This U.S. patent application claims priority under 35 U.S.C. § 119(e) to U.S. Provisional Application 63/329,352, filed on Apr. 8, 2022, and U.S. Provisional Application 63/422,763, filed on Nov. 4, 2022. The disclosures of these prior applications are considered part of the disclosure of this application and are hereby incorporated by reference in their entireties.

### TECHNICAL FIELD

This disclosure relates to machine learning identification, classification, and quantification of tertiary lymphoid structures, e.g., in tumor biopsy specimens.

### BACKGROUND

Tertiary lymphoid structures (TLS) (e.g., tertiary lymphoid organ or ectopic lymphoid follicle) are ectopic lymphoid tissue composed of B-cells, T-cells, and supportive cells that develop in non-lymphoid organs and are often found in tumors. TLS support differentiation of naïve T cells to effector and memory T cells and frequently develop in areas of chronic inflammation. In the clinical pathology setting, TLS have been observed, but are not currently assessed for diagnostic pathology, or to guide therapy. Studies have shown associations between TLS and immuno-oncology (IO) treatment outcomes across multiple indications (e.g., as described in Sautes-Fridman, et al, 2019, Nat Rev Cancer 19:307and Vanhersecke, et al, “Mature tertiary lymphoid structures predict immune checkpoint inhibitor efficacy in solid tumors independently of PD-L1 expression,” Nat Cancer, 2021). Presence of TLS in various tumors shows an association with outcomes in the non-IO setting, and recently TLS have been shown to be predictive of response to IO treatment in melanoma, bone sarcoma, and RCC. See e.g., Cabrita, et al, 2020, Nature 577:561, Petitprez, et al, 2020, Nature 577:556, Helmink, et al, 2020, Nature 577:549, Bruno, N&V, 2020, Nature 577:474, Sautes-Fridman, et al, 2019, Nat Rev Cancer 19:307. In the research setting, TLS have been assessed by manual visual methods based on hematoxylin and eosin stain (H&E) and immunohistochemistry (IHC) staining. Image analysis of IHC or immunofluorescent (IF) staining has been used for quantification. These correlations are dependent on TLS maturity and localization within the tumor microenvironment (TME).

### SUMMARY

One aspect of the disclosure provides a computer-implemented method that when executed on data processing hardware causes the data processing hardware to perform operations that include receiving an input histology image for a patient diagnosed with cancer. The input histology image includes a plurality of image pixels. The operations also include processing, using a cell classification model, the input histology image to generate one or more lymphocyte density maps within the input histology image, and performing morphological image processing on the one or more lymphocyte density maps to identify one or more TLS regions within the input histology image. Each TLS region is represented by a respective cluster of lymphocyte cells. For each corresponding TLS region of the one or more TLS regions identified in the input histology image, the operations also include extracting, from the respective cluster of lymphocyte cells representing the corresponding TLS region, a respective set of TLS features, and processing, using a TLS classification model, the respective set of TLS features to classify the corresponding TLS region as one of a first TLS maturation state, a second TLS maturation state, or a third TLS maturation state.

Implementations of the disclosure may include one or more of the following optional features. In some implementations, the operations also include processing, using a tumor detection model, the input histology image to identify a tumor region within the input histology image. Here, processing the input histology image to generate the one or more lymphocyte density maps may include processing, using the cell classification model, the input histology image by performing single-cell imaging analysis on the tumor region identified within the input histology image to generate the one or more lymphocyte density maps. In these implementations, the tumor detection model may be trained by obtaining a plurality of image tiles rasterized from a set of whole-slide histopathology images, each image tile manually annotated as including a tumor or a non-tumor, and training, using a neural network, the tumor detection model on the plurality of image tiles to teach the tumor detection model to learn how to identify tumor regions within histology images.

In some examples, the cell classification model is trained by obtaining a plurality of image patches and training, using a neural network, the cell classification model on the plurality of image patches to teach the cell classification model to learn how to classify individual cells in histology images as tumor cells, lymphocyte cells, or non-malignant cells. Each image patch includes a corresponding plurality of human cells and manual annotations that label each human cell as a tumor cell, a lymphocyte cell, or a non-malignant cell.

In some implementations, the TLS classification model is trained by obtaining a training dataset comprising a plurality of training histology images, wherein each training histology image includes a tumor microenvironment and has manual annotations. The manual annotations identify one or more TLS regions in the training histology image, and for each corresponding TLS region, a ground-truth TLS maturation state indicating that the corresponding TLS region includes a first TLS maturation state, a second TLS maturation state, or a third TLS maturation state. Each TLS region is represented by a respective cluster of lymphocyte cells. In these implementations, the TLS classification model is further trained by, for each TLS region, extracting, from the respective cluster of lymphocyte cells representing the TLS region, a respective set of training TLS features, and training the TLS classification model on the respective set of training TLS features extracted for each TLS region to teach the TLS classification model to learn how to predict the ground-truth TLS grade for each corresponding TLS region. Training the TLS classification model may include training the TLS classification model using a classification and regression trees (CART) algorithm.

The first TLS maturation state may include a dense aggregate of at least a threshold number of lymphocytes that do not contain high endothelial venules or germinal centers. The second TLS maturation state may include an immature TLS including a dense aggregate of at least the threshold number of lymphocytes that contain high endothelial venules and do not contain any germinal centers. The third TLS maturation state may include a mature TLS including a dense aggregate of at least the threshold number of lymphocytes that contain high endothelial venules and germinal centers. The respective set of TLS features extracted from the respective cluster of lymphocyte cells may include an area of the corresponding TLS region, a roundness of the corresponding TLS region, and a skewness of the corresponding TLS region.

In some examples, the operations further include, for each corresponding TLS region of the one or more TLS regions identified in the input histology image, generating a respective pixel mask that highlights at least a perimeter of the corresponding TLS region, generating an output image that augments the input histology image by overlaying the respective pixel mask generated for each of the TLS regions onto the input histology image, and providing, for display on a screen in communication with the data processing hardware, the output image. In these examples, the respective pixel mask generated for each corresponding TLS region classified as the first maturation state includes a first pixel mask, the respective pixel mask generated for each corresponding TLS region classified as the second maturation state includes a second pixel mask that is visually distinguishable from the second pixel mask, and the respective pixel mask generated for each corresponding TLS region classified as the third maturation state includes a third pixel mask that is visually distinguishable from the first pixel mask and the second pixel mask.

In some implementations, the operations also include determining an overall TLS score for the input histology image based on the TLS maturation states for the one or more TLS regions identified in the histology image and the TLS features extracted from the one or more TLS regions identified in the histology image. In these implementations, the operations may also include determining a treatment recommendation to treat the patient using immunotherapy based on the overall TLS score. Here, the immunotherapy may include at least one of PD-1 inhibitor or a PD-L1 inhibitor. The operations may also include determining a predictive score of the patient's response to immunotherapy based on the TLS maturation states for the one or more TLS regions identified in the histology image and the TLS features extracted from the one or more TLS regions identified in the histology image.

Another aspect of the disclosure provides a system that includes data processing hardware and memory hardware in communication with the data processing hardware. The memory hardware stores instructions that when executed on the data processing hardware causes the data processing hardware to perform operations that include receiving an input histology image for a patient diagnosed with cancer. The input histology image includes a plurality of image pixels. The operations also include processing, using a cell classification model, the input histology image to generate one or more lymphocyte density maps within the input histology image, and performing morphological image processing on the one or more lymphocyte density maps to identify one or more TLS regions within the input histology image. Each TLS region is represented by a respective cluster of lymphocyte cells. For each corresponding TLS region of the one or more TLS regions identified in the input histology image, the operations also include extracting, from the respective cluster of lymphocyte cells representing the corresponding TLS region, a respective set of TLS features, and processing, using a TLS classification model, the respective set of TLS features to classify the corresponding TLS region as one of a first TLS maturation state, a second TLS maturation state, or a third TLS maturation state.

This aspect may include one or more of the following optional features. In some implementations, the operations also include processing, using a tumor detection model, the input histology image to identify a tumor region within the input histology image. Here, processing the input histology image to generate the one or more lymphocyte density maps may include processing, using the cell classification model, the input histology image by performing single-cell imaging analysis on the tumor region identified within the input histology image to generate the one or more lymphocyte density maps. In these implementations, the tumor detection model may be trained by obtaining a plurality of image tiles rasterized from a set of whole-slide histopathology images, each image tile manually annotated as including a tumor or a non-tumor, and training, using a neural network, the tumor detection model on the plurality of image tiles to teach the tumor detection model to learn how to identify tumor regions within histology images.

In some examples, the cell classification model is trained by obtaining a plurality of image patches and training, using a neural network, the cell classification model on the plurality of image patches to teach the cell classification model to learn how to classify individual cells in histology images as tumor cells, lymphocyte cells, or non-malignant cells. Each image patch includes a corresponding plurality of human cells and manual annotations that label each human cell as a tumor cell, a lymphocyte cell, or a non-malignant cell.

In some implementations, the TLS classification model is trained by obtaining a training dataset comprising a plurality of training histology images, wherein each training histology image includes a tumor microenvironment and has manual annotations. The manual annotations identify one or more TLS regions in the training histology image, and for each corresponding TLS region, a ground-truth TLS maturation state indicating that the corresponding TLS region includes a first TLS maturation state, a second TLS maturation state, or a third TLS maturation state. Each TLS region is represented by a respective cluster of lymphocyte cells. In these implementations, the TLS classification model is further trained by, for each TLS region, extracting, from the respective cluster of lymphocyte cells representing the TLS region, a respective set of training TLS features, and training the TLS classification model on the respective set of training TLS features extracted for each TLS region to teach the TLS classification model to learn how to predict the ground-truth TLS grade for each corresponding TLS region. Training the TLS classification model may include training the TLS classification model using a classification and regression trees (CART) algorithm.

The first TLS maturation state may include a dense aggregate of at least a threshold number of lymphocytes that do not contain high endothelial venules or germinal centers. The second TLS maturation state may include an immature TLS including a dense aggregate of at least the threshold number of lymphocytes that contain high endothelial venules and do not contain any germinal centers. The third TLS maturation state may include a mature TLS including a dense aggregate of at least the threshold number of lymphocytes that contain high endothelial venules and germinal centers. The respective set of TLS features extracted from the respective cluster of lymphocyte cells may include an area of the corresponding TLS region, a roundness of the corresponding TLS region, and a skewness of the corresponding TLS region.

In some examples, the operations further include, for each corresponding TLS region of the one or more TLS regions identified in the input histology image, generating a respective pixel mask that highlights at least a perimeter of the corresponding TLS region, generating an output image that augments the input histology image by overlaying the respective pixel mask generated for each of the TLS regions onto the input histology image, and providing, for display on a screen in communication with the data processing hardware, the output image. In these examples, the respective pixel mask generated for each corresponding TLS region classified as the first maturation state includes a first pixel mask, the respective pixel mask generated for each corresponding TLS region classified as the second maturation state includes a second pixel mask that is visually distinguishable from the second pixel mask, and the respective pixel mask generated for each corresponding TLS region classified as the third maturation state includes a third pixel mask that is visually distinguishable from the first pixel mask and the second pixel mask.

In some implementations, the operations also include determining an overall TLS score for the input histology image based on the TLS maturation states for the one or more TLS regions identified in the histology image and the TLS features extracted from the one or more TLS regions identified in the histology image. In these implementations, the operations may also include determining a treatment recommendation to treat the patient using immunotherapy based on the overall TLS score. Here, the immunotherapy may include at least one of PD-1 inhibitor or a PD-L1 inhibitor. The operations may also include determining a predictive score of the patient's response to immunotherapy based on the TLS maturation states for the one or more TLS regions identified in the histology image and the TLS features extracted from the one or more TLS regions identified in the histology image.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1 is a schematic view of an example system for identifying, classifying, and quantifying tertiary lymphoid structures (TLS) in histology images of tumor microenvironments.

FIGS. 2A-2K illustrates a plurality of tables that list exemplary TLS features.

FIG. 3A is a schematic view of an example training process for training a TLS classification model.

FIG. 3B is a schematic view of an example training process for training a tumor detection model.

FIG. 3C is a schematic view of an example training process for training a cell classification model.

FIG. 4 is a flowchart of an example arrangement of operations for a method of identifying, classifying, and quantifying TLS in histology images of tumor microenvironments.

FIGS. 5A and 5B are example confusion matrices comparing accuracies between a TLS classification model and pathologists.

FIGS. 6A-6C are example plots comparing performance between the TLS classification model and pathologists.

FIG. 7 illustrates example input histology images and the corresponding classified TLS maturation states.

FIG. 8 illustrates an example input histology image representing a mature TLS maturation state and a corresponding output image that includes a respective pixel mask.

FIG. 9 illustrates an example input histology image representing an immature TLS maturation state and a corresponding output image that includes a respective pixel mask.

FIG. 10 illustrates an example input histology image representing a lymphoid aggregate TLS maturation state and a corresponding output image that includes a respective pixel mask.

FIG. 11 illustrates an example output image that includes TLS regions corresponding to each of a mature TLS maturation state, an immature TLS maturation state, and a lymphoid aggregate TLS maturation state.

FIGS. 12-16 illustrate example untransformed output images and transformed output images.

FIG. 17 is a schematic view of a process flow diagram for validating extracted TLS features using transcriptomic analysis correlation.

FIG. 18 illustrates an example table of a 12-chemokine gene signature.

FIG. 19 illustrates an example feature table.

FIGS. 20A-20C illustrate example graphical representations of correlation data.

FIGS. 21A-21D illustrate example graphical representations of correlation diagrams that validate the extracted TLS features 140.

FIG. 22 is a schematic view of an example computing device that may be used to implement the systems and methods described herein.

Like reference symbols in the various drawings indicate like elements.

### DETAILED DESCRIPTION

Tertiary lymphoid structures (TLSs) are ectopic lymphoid organs that develop in nonlymphoid tissues, such as sites of chronic inflammation and tumors. TLS are vascularized lymphoid structures that develop in benign and tumor tissues with chronic inflammation. TLS are highly organized structures that are similar to secondary lymphoid structures (e.g., lymph nodes). TLS can be composed of B-cell zones containing active germinal centers, surrounding T-cell zones that contain various types of dendritic cells (DCs), T-cells, high endothelial venules (HEVs), and/or other supportive cells within a structural matrix. Unlike lymph nodes, TLS lack fibrous capsules and are directly exposed to a tumor microenvironment (TME). TLS are more abundant in the invasive margin/stroma as compared to tumor cores. The presence of TLS is associated with favorable outcomes in treatment of multiple indications (e.g., treatment of melanoma with nivolumab or nivolumab and ipilimumab). TLS structures can be classified as lymphoid aggregates (LA) (i.e., a first maturation state), immature TLS (imTLS) (e.g., Grade 1) (i.e., a second maturation state), or mature TLS (mTLS) (e.g., Grade 2) with the presence of a germinal center (GC) (i.e., a third maturation state). In some cases, there is no TLS (e.g., Grade 0). While the biological mechanisms behind their formation are incompletely understood, TLSs are known to play an important role in antitumor immune response. For instance, the presence of TLSs has been associated with a favorable prognosis and improved response to immunotherapy across many cancer types.

The conventional approach to TLS detection in patients is through the technique of tissue staining for markers of immune cell lineages by multiplex immunohistochemistry or immunofluorescence techniques. However, multiplex imaging is not routinely applicable given its cost, high complexity, small field of view, and difficulty to scale, which limit its use to research settings. On the other hand, hematoxylin-eosin (H&E)-staining is widely available and remains the clinical standard in histopathology. Evaluating H&E-stained slides based on pathologist assessment is time and labor intensive, and manual and qualitative evaluations performed manually by pathologists are often inaccurate and subject to interobserver variability.

Implementations herein are directed toward leveraging machine learning techniques that use deep learning to train models to learn how to detect the presence of TLS regions in H&E-stained histology images and classify each of the TLS regions into one of three TLS maturation states. A first maturation state includes a dense aggregate of at least a threshold number of lymphocytes that do not contain high endothelial venules or germinal centers. In some examples, the threshold number is equal to 100. The second maturation state includes an immature TLS associated with a dense aggregate of at least the threshold number of lymphocytes that contain high endothelial venules and do not contain any germinal centers. The third maturation state includes a mature TLS associated with a dense aggregate of at least the threshold number of lymphocytes that contain high endothelial venules and germinal centers. More specifically, implementations include using a cell classification model to process an input histology image (e.g., H&E-stained histology image) to generate one or more lymphocyte density maps, performing morphological image processing on the one or more lymphocyte density maps to identify one or more TLS regions within the input histology image where each TLS region is represented by a respective cluster of lymphocyte cells, and for each corresponding TLS region, extracting, from the respective cluster of lymphocyte cells representing the corresponding TLS region, a respective set of TLS features. Thereafter, a trained TLS classification model receives the respective set of TLS features extracted for each corresponding TLS region to classify the corresponding TLS region as one of the first TLS maturation state, the second TLS maturation state, or the third TLS maturation state.

Implementations herein are further directed toward calculating a TLS score for the input histology image based on TLS maturation states output from TLS classification model and the TLS features for the TLS regions identified in the input histology image. A TLS scorer may determine a total area of the tumor area and also a respective TLS score for each of the three TLS maturation states that is based on the respective total TLS area of the TLS regions classified for each of the three TLS maturation states. The TLS scorer may then compute an overall TLS score for the patient associated with the input histology image based on a linear weighted sum of each respective total TLS area divided by the tumor area. Described in greater detail below, the overall TLS score may be used to predict various prognostic values for the patient such as predicting survival outcomes such as overall survival and progression-free survival. That is, higher overall TLS scores are indicative of significantly improved overall survival and progression-free survival compared to lower overall TLS scores. As such, overall TLS scores may be used to predict prognostic outcomes in lieu of using tumor stage predictions and/or prognostic outcomes predicted using tumor stage/grade may be further refined by the overall TLS scores.

For each corresponding TLS region of the one or more TLS regions identified in the input histology image, an image augmenter may generate a respective pixel mask that highlights at least a perimeter of the corresponding TLS region and then generate an output image that augments the input histology image by overlaying the respective pixel mask generated for each of the TLS regions onto the input histology image. The output image generated by the image augmenter may be provided for display on a screen for a healthcare professional (HCP) to view. Here, the image augmenter receives the classification outputs from the TLS classification model and generates visually different respective pixel masks for each of the three different TLS maturation states. For instance, the pixel mask generated for TLS regions classified as the first maturation state may include a first color, the pixel mask generated for TLS regions classified as the second maturation state may include a different second color, and the pixel mask generated for the TLS regions classified as the third maturation state may include a third color different than the first and second colors. In some examples, the pixel generated for the TLS regions classified as the third maturation state highlight at least the perimeter of the corresponding TLS region and further highlight an area of pixels encompassed by the germinal center.

Implementations herein are further directed toward a training process for training the TLS classification model. Here, the training process obtains a training dataset that includes a plurality of training histology images each containing a tumor microenvironment and including manual annotations from pathologists. The manual annotations identify the presence of TLS regions in each training histology image where each TLS region is represented by a respective cluster of lymphocyte cells. The manual annotations further identify a ground-truth TLS maturation state for each corresponding TLS region indicating that the corresponding TLS region includes the first TLS maturation state, the second TLS maturation state, or the third maturation state. Next, the training process extracts, from the respective cluster of lymphocyte cells representing each TLS region, a respective set of training TLS features that may include area of the TLS region, roundness (i.e., the ratio of the area of TLS region multiplied by 4 pi to a square of a perimeter of the TLS region), and skewness of the density of the respective cluster of lymphocyte cells representing each TLS region. Based on the respective set of training TLS features extracted for each TLS region, the training process trains the TLS model using a classification and regression trees (CART) algorithm to learn how to predict the ground-truth TLS grade for each corresponding TLS region.

Notably, the cell classification model is trained to learn how to classify individual cells in histology images as tumor cells, lymphocyte cells, or non-malignant cells. As used herein, lymphocyte cells may include T-cells and B-cells. The cell classification model may be trained using a Mask R-CNN deep learning model to learn how to segment and classify individual nuclei into tumor cells, lymphocytes, and other nonmalignant cells.

In some examples, image pre-processing is performed on the input histology image by using a tumor detection model to process the input histology image to identify a tumor region within the input histology image such that the cell classification model is used to perform single-cell image analysis on the tumor region identified within the input histology to generate the one or more lymphocyte density maps. The tumor detection model may be trained on a plurality of image tiles rasterized from a set of whole-slide histopathology images with each image tile manually annotated as including a tumor or a non-tumor. More specifically, a deep learning neural network trains the tumor detection model on the plurality of image tiles to teach the tumor detection model to learn how to identify tumor regions within histology images. The deep learning neural network may include a ResNet18 deep learning model.

Advantageously, the deep learning-based single-cell analysis techniques disclosed herein provide the ability to accurately identify, classify, and quantify the presence of TLS regions from H&E-stained whole-slide images without incurring any of the drawbacks of other techniques that adopt patch- or tile-based approaches for image analysis. Since TLSs are highly variable in size, density, and morphology, there are significant challenges using traditional patch-based approaches for identifying and interpreting TLS regions. As will become apparent, the techniques disclosed therein include quantifying the spatial distribution of lymphocytes to thereby provide an accurate and interpretable model for classification of TLSs according to their maturation states.

Similarly, manual and qualitative assessment of TLSs performed by pathologists lack automated enumeration and quantitative characterization of TLS. By the same notion, such manual and qualitative assessment of TLSs performed by pathologists are found to be inaccurate and subject to interobserver variability when assessed on H&E-stained slides. See Buisseret L, Desmedt C, Garaud S, et al. Reliability of tumor-infiltrating lymphocyte and tertiary lymphoid structure assessment in human breast cancer. Mod Pathol. 2017; 30(9):1204-1212. doi:10.1038/modpathaol.2017.43.

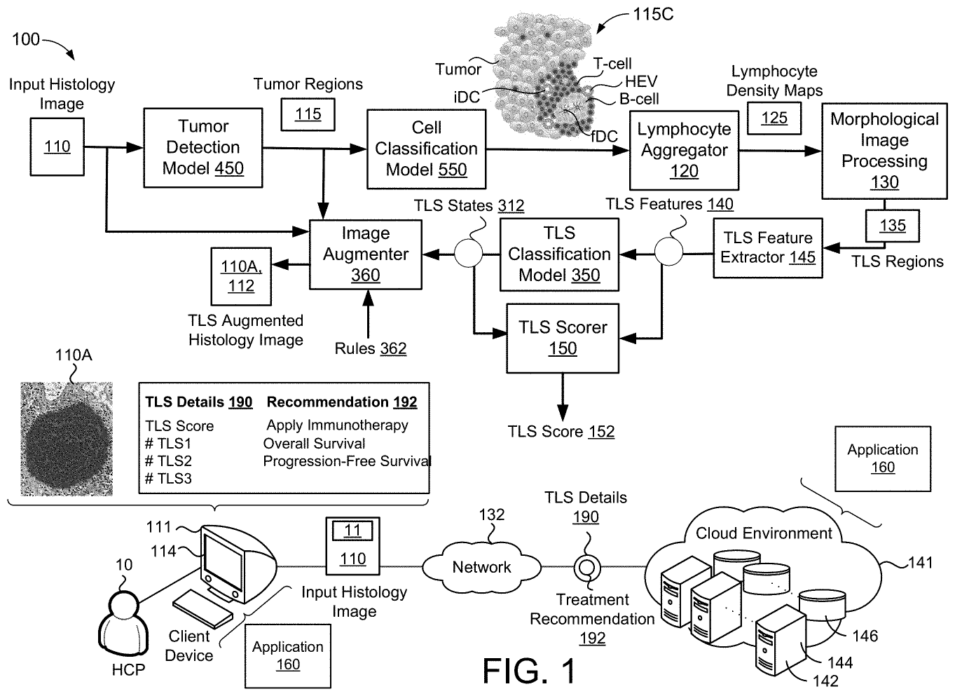

Referring to FIG. 1, in some implementations, a system 100 includes a client device 111 inputting a histology image 110 for a patient diagnosed with cancer to a TLS classification model 350 for identifying, classifying, and quantifying the presence of TLS regions within the histology image for use as a predictive biomarker of immune-checkpoint inhibitor (ICI) efficacy and prognostic outcome. The input histology image 110 may optionally include metadata 11 that includes information such as a type of cancer the patient is diagnosed with, a stage/grade of a tumor, and/or patient demographic information. The input histology image 110 may include a hematoxylin and eosin (H&E)-stained whole slide image (WSI). The input histology image 110 includes a plurality of image pixels. The input histology image 110 characterizes a human tumor biopsy specimen. The input histology image 110 may contain a tumor microenvironment for any number of cancers including, without limitation, bladder cancer (BLCA), breast cancer (BRCA), stomach adenocarcinoma (STAD), lung adenocarcinoma (LUAD) (e.g., non-small cell lung cancer adenocarcinoma (NSCLC-AD)), and/or lung squamous cell carcinoma (LUSC) (e.g., non-small cell lung cancer squamous (NSCLC-SQ)).

The client device 111 is associated with a user 10 such as a healthcare professional (HCP), who may communicate, via a network 132, with a remote system 141. The remote system 141 may be a distributed system (e.g., cloud environment) having scalable/elastic resources 142. The resources 142 include computing resources 144 (e.g., data processing hardware) and/or storage resources 146 (e.g., memory hardware). In some implementations, the remote system 141 executes a TLS identification and quantification application 160 (also referred to as simply “application 160”) configured to execute the TLS classification model 450 in addition to other components such as a tumor detection model 450, a cell classification model 550, a lymphocyte aggregator 120, a morphological image processing module 130, a TLS extractor 145, a TLS scorer 150, and an image augmenter 360. Here, the client device 111 may access the application 160 running on the remote system 141 and input, via a graphical user interface (GUI) executing on the client device 111, the histology input image 110 to the TLS classification model 350. The GUI may be displayed to the user 10 via a screen 114 of the client device 111. The client device 111 may additionally or alternatively execute the application 160 to implement the ability to run any combination of the TLS classification model 350 and/or other components on the client device 111 for identifying, classifying, and quantifying the presence of TLS regions 135 within the histology image 110.

The TLS identification and quantification application 160 may ascertain TLS details 190 and/or a treatment recommendation 192 based on the identified TLS regions 135 classified and quantified using the TLS classification model 350. The application 160 may return the TLS details 190 and/or the treatment recommendation 192 to the client device 111 to cause the client device to display the TLS details 190 and/or the treatment recommendation 192 on the screen 114 of the client device 111. The TLS details 190 may include, without limitation, an overall TLS score 152 for the input histology image 110 as well as other details such as the number of TLS regions associated with a first maturation state (e.g., TLS1) classified by the TLS classification model 350, the number of TLS regions associated with a second maturation state (e.g., TLS2) classified by the TLS classification model 350, and the number of TLS regions associated with a third maturation state (e.g., TLS3) classified by the TLS classification model 350. Here, the first maturation state includes a dense aggregate of at least a threshold number of lymphocytes that do not contain high endothelial venules or germinal centers. In some examples, the threshold number is equal to 100. The second maturation state includes an immature TLS associated with a dense aggregate of at least the threshold number of lymphocytes that contain high endothelial venules and do not contain any germinal centers. The third maturation state includes a mature TLS associated with a dense aggregate of at least the threshold number of lymphocytes that contain high endothelial venules and germinal centers. The TLS details 190 provided for display on the screen 114 may further include an output image 110A augmenting the input histology image 110 by overlaying a respective pixel mask 112 generated for each of the TLS regions onto the input histology image 110. The treatment recommendations 192 may indicate instructions to apply (or not apply) immunotherapy to the patient for treating the patient. For instance, the immunotherapy may include a PD-1 inhibitor (e.g., an anti-PD-1 antibody) or a PD-L1 inhibitor (e.g., an anti-PID-L1 antibody). In one example, the immunotherapy includes the immune checkpoint inhibitor drug nivolumab.

The treatment recommendations 192 may further include prognostic outcomes predicted for the patient based on the TLS details 190 such as overall survival (OS) (i.e., in months), progression-free survival (PFS) (in months). The treatment recommendations 192 may show OS and/or PFS predictions for immunotherapy treatment contrasted by OS and/or PFS predictions without immunotherapy. The prognostic outcomes predicted by the application 160 may inform a patient, healthcare provider, and/or relatives of the patient for making better testing and treatment decisions for a specific health condition is diagnosed with, or for making risk-stratifications for therapeutic trials.

In some examples, the input histology image 110 undergoes initial image preprocessing to ensure sufficient image quality. The input histology image may include a 40× magnification. However, WSI slides scanned at lower magnification (e.g., 20×) may be used. To minimize the influence of image artifacts, the image preprocessing may down-sample the whole-slide images by a factor of 32 and apply appropriate color factors to remove regions with pen marks, folding, and blurring artifacts.

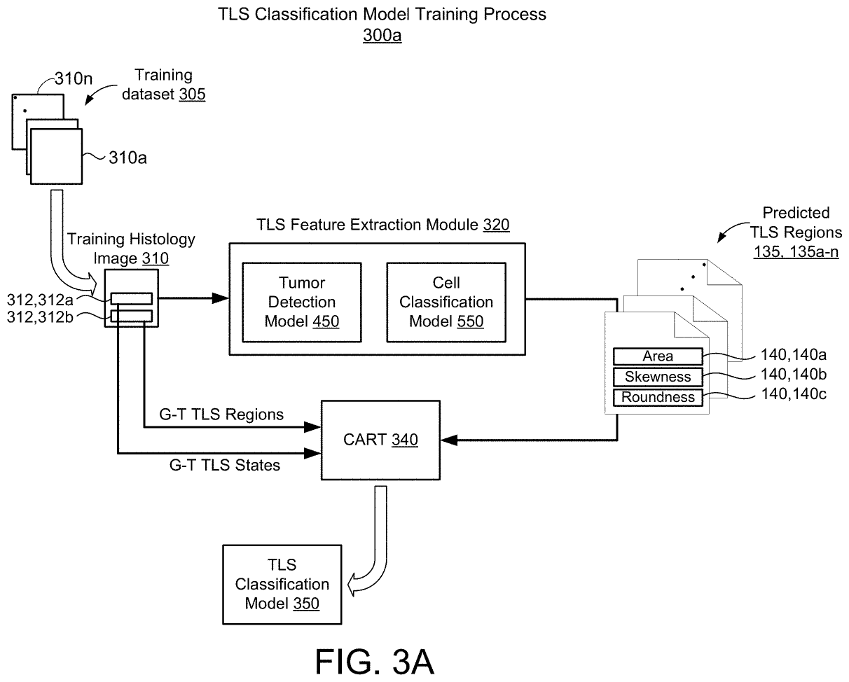

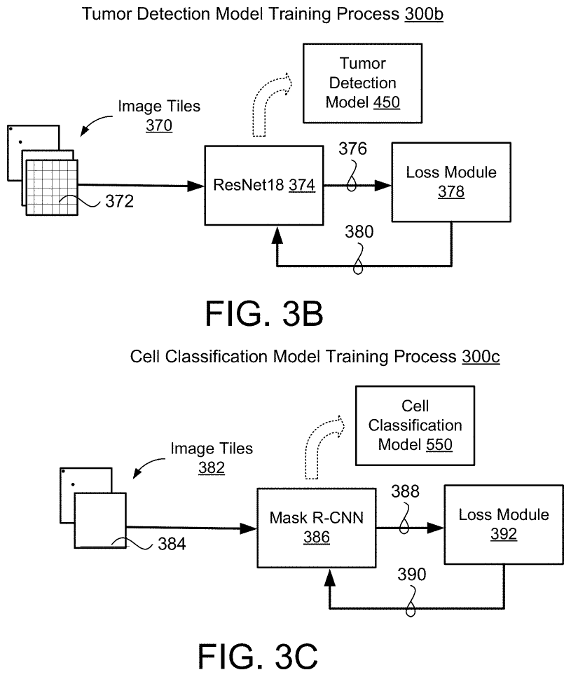

In the example shown, a tumor detection model 450 processes the input histology image 110 to identify one or more tumor regions 115 within the input histology image 110. Each tumor region 115 may be represented by a corresponding group of pixels where the tumor region 115 is located input histology image 110. Notably, since only TLS within or around a tumor region 115 are relevant, the tumor detection model 450 may segment cancerous tissue from normal tissue, enabling subsequent processing for TLS identification and quantification to be focused on the tumor regions 115 in the input histology image 110. The tumor detection model 450 may include a pre-trained indication-specific tissue segmentation model configured to process the input histology image 110 to distinguish cancer, cancer-associated stroma, and necrosis from normal tissue. FIG. 3B shows an example tumor detection model training process 300b that may be used to train the tumor detection model 450. The training process 300b obtaining a plurality of image tiles 370 rasterized from a set of whole-slide histopathology images. The histopathology images may include publicly available and previously annotated H&E-stained WSIs from patients with colorectal cancer and stomach cancer. Each image tile 370 may include manual annotations 372 indicating locations of tumor regions and non-tumor regions (including adipose tissue, mucus, stroma, or muscle) within the corresponding whole-slide histopathology image. The image tiles may include 512×512 image tiles at 0.5 micrometers per image pixel. The training process 300b includes training, using a neural network 374, the tumor detection model 450 on the plurality of image tiles 370 to teach the tumor detection model 450 to learn how to identify tumor regions within histology images. In some examples, the neural network 374 includes a ResNet18 deep learning network and a loss module 378 computes training losses 380 based on predictions 376 output by the ResNet 18 network relative to ground-truth annotations 372. The training process 300b may update parameters of the REsNet 18 based on the training losses 380 until the parameters of the ResNet 18 converge to obtain the trained tumor detection model 450. The loss module 378 may employ a cross-entropy loss function and counteract overfitting by applying L2-regularization. The training process 300b may expand tumor segmentation via image dilation by 0.5 mm to include an invasive margin. The training process may further apply horizontal/vertical flipping and translation to augment the image tiles 370 used for training.

Referring back to FIG. 1, after the tumor detection model 450 identifies the tumor region 115, the cell classification model 550 processes the input histology image 110 by performing single-cell imaging analysis on the tumor region 115 (i.e., on the image pixels corresponding to the tumor region 115) identified within the input histology image 110 to generate a classified tumor region 115C. Namely, the single-cell imaging analysis performed by the cell classification model 550 classifies individual cells/nuclei as tumor cells, lymphocyte cells (i.e., B-Cells and T-Cells, dendritic cells (DCs), high endothelial venules (HEVs)), and non-malignant cells. As used herein, the trained cell classification model 550 functions as a lymphocyte mask for classifying which cells in the tumor region 115 include lymphocytes. As such, the classified tumor region 115C may correspond to the lymphocyte mask identifying all the lymphocyte cells classified and segmented by the cell classification model 550 in the tumor region 115 within the input histology image 110. Thereafter, the application 160 executes a lymphocyte aggregator 120 that processes the classified tumor region 115C output by the cell classification model 550 to count a number of lymphocytes per unit square on a predefined grid (e.g., 16×16 μm² grid) to generate one or more lymphocyte density maps 125 within the input histology image 110.

FIG. 3C shows an example cell classification model training process 300c that may be used to train the cell classification model 550 on a plurality of image patches 382. Each image patch (i.e, image tile) 382 characterizes a corresponding plurality of human cells and is manually annotated to label each human cell as a tumor cell, or lymphocyte cell, or a non-malignant cell. The plurality of image patches may include 1,358 image patches from 66 patients in a publicly available dataset with manual annotations 384 containing 17,582 tumor cells, 22,550 lymphocyte cells, and 10,675 other non-malignant cells. The training process 300c includes training, using a neural network 386, the cell classification model 550 on the plurality of image patches 382 to teach the cell classification model 550 to learn how to classify individual cells in histology images as tumor cells, lymphocyte cells, or non-malignant cells. In some examples, a neural network 386 includes a Mask R-CNN deep learning network and a loss module 392 computes training losses 390 based on predictions 388 output by the Mask R-CNN network 386 relative to ground-truth annotations 384. The training process 300b may update parameters of the Mask R-CNN based on the training losses 392 until the parameters of the Mask R-CNN converge to obtain the trained cell classification model 550. As used herein, the trained cell classification model 550 functions as a lymphocyte mask for classifying which cells in the tumor region 115 include lymphocytes. The loss module 392 may update the Mask R-CNN via the training losses 392 using stochastic gradient descent techniques. The training process may further apply horizontal/vertical flipping and translation to augment the image patches 382 used for training.

Referring back to FIG. 1, in some implementations, the TLS identification and quantification application 160 performs morphological image processing 130 on the one or more lymphocyte density maps 125 to identify one or more TLS regions 135 within the input histology image 110. Notably, each TLS region represents a respective cluster of lymphocyte cells. The morphological image processing 130 may indicate the pixel locations that correspond to each TLS region 135 identified within the input histology image 110. Each TLS region 135 may correspond to a TLS mask. In some examples, the morphological image processing 130 performed on the lymphocyte density maps 125 applies thresholding to exclude lymphocyte clusters having areas that are less than a predefined threshold area from being identified as TLS regions. The predefined threshold area may be equal to 0.0384 mm².

For each TLS region 135 identified, the application 160 executes a TLS feature extractor 145 configured to extract, from the respective cluster of lymphocyte cells representing the corresponding TLS region 135, a respective set of TLS features 140. The set of TLS features 140 may include human interpretable features (HIFs) associated with the TLS region 135. In some examples, a portion of the TLS features include sample level features including at least one of a summary count, an area, a shape, or a location of the corresponding TLS region 135. The TLS features 140 extracted from the respective cluster of lymphocyte cells representing the corresponding TLS region 135 may include an area of the TLS region 135, a roundness of the TLS region 135 (i.e., the ratio of the area of TLS region 135 multiplied by 4 pi to a square of a perimeter of the TLS region 135), and skewness of the density of the respective cluster of lymphocyte cells representing the TLS region 135). The TLS features 140 may additionally or alternatively at least one of an area of germinal center within object in tissue, an area of object in tissue, a centroid x of object in tissue, a centroid y of object in tissue, a longest distance of object from tumor, a perimeter of object in tissue, a shortest distance of object from tumor, a total germinal center within object in tissue, or an area prop germinal center within object over object in tissue. Some of the TLS features 140 may include sample level features including one or more of an area of the TLS region 135, a total count of lymphocyte cells, area proportion, count proportion, maximum area, maximum longest distance from tumor, maximum perimeter, maximum shortest distance from tumor, maximum total area, maximum total count, mean area, mean longest distance from tumor, mean perimeter, mean shortest distance from tumor, mean total area, mean total count, median area, median longest distance from tumor, median perimeter, median shortest distance from tumor, median total area, median total count, minimum area, minimum longest distance from tumor, minimum perimeter, minimum shortest distance from tumor, minimum total area, or minimum total count.

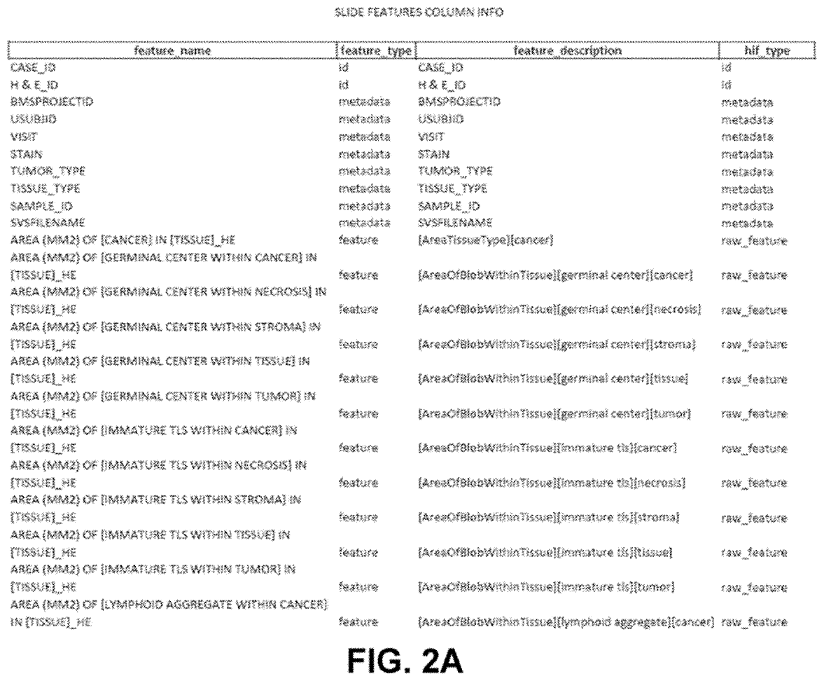

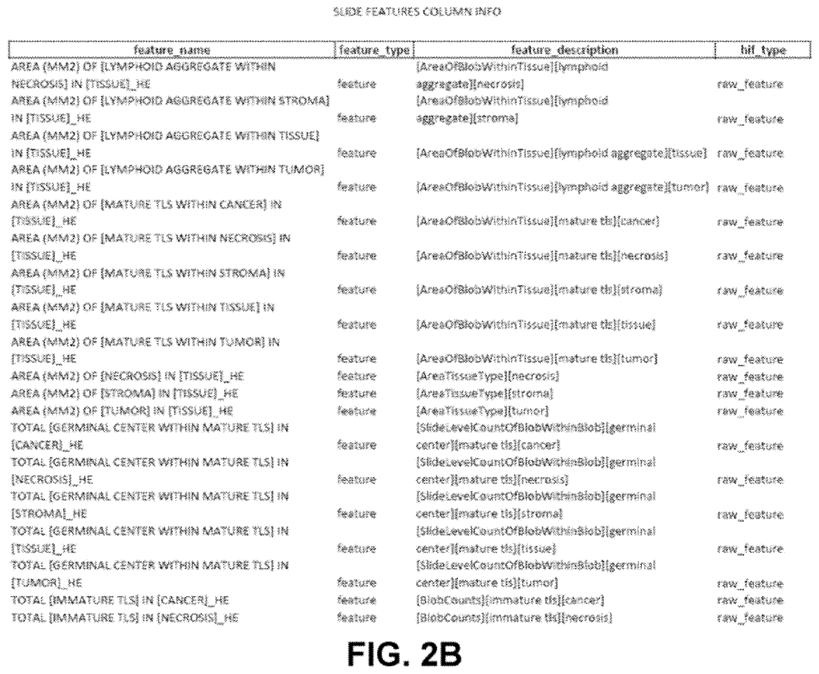

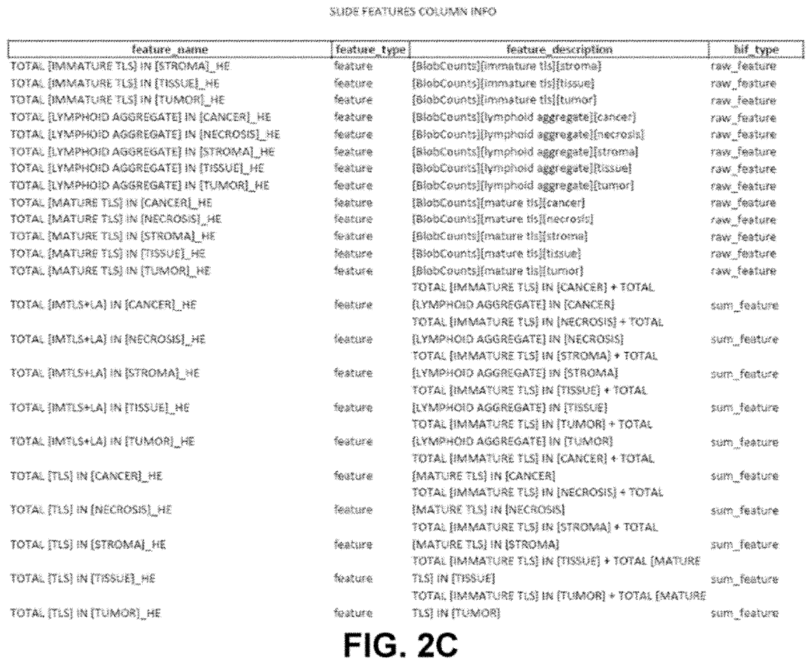

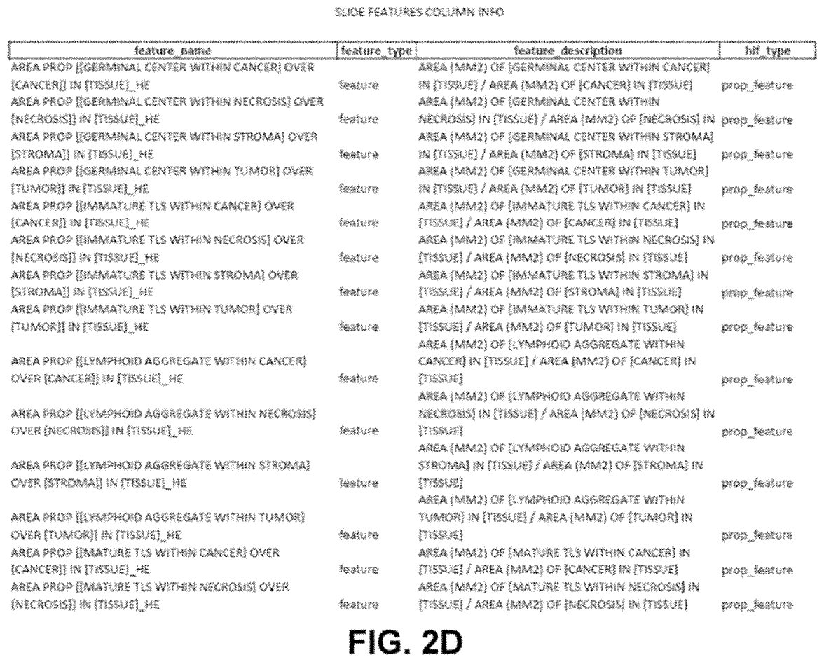

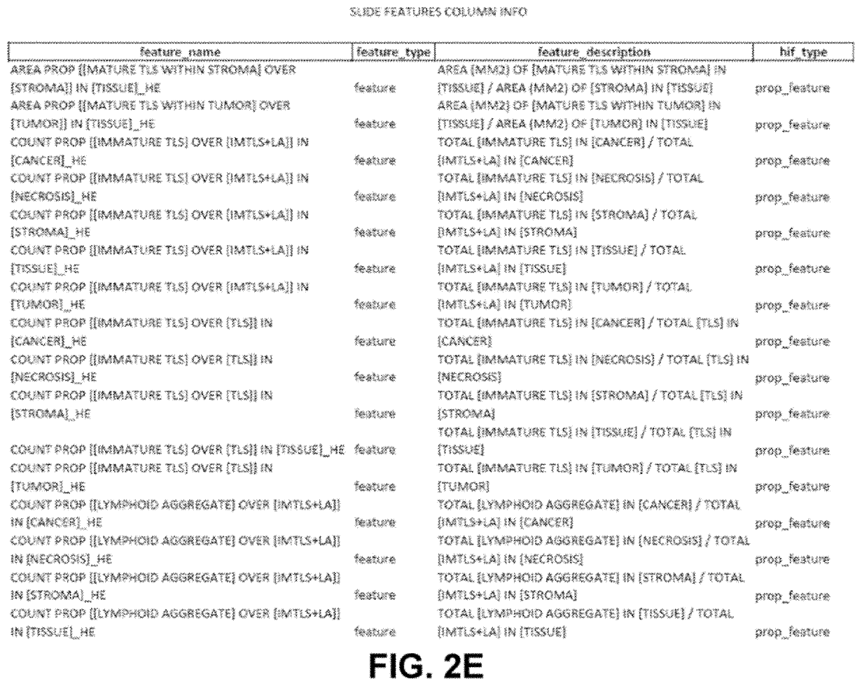

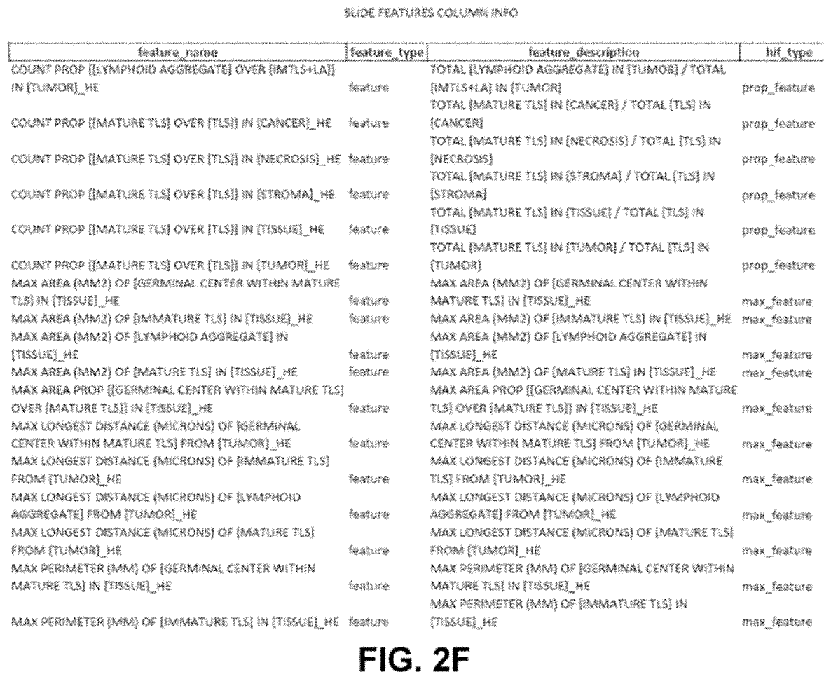

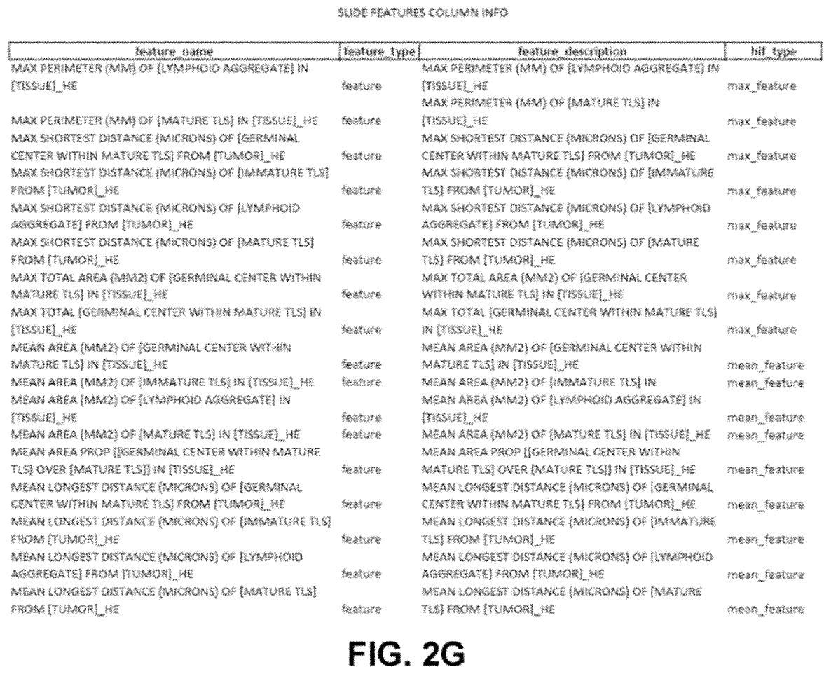

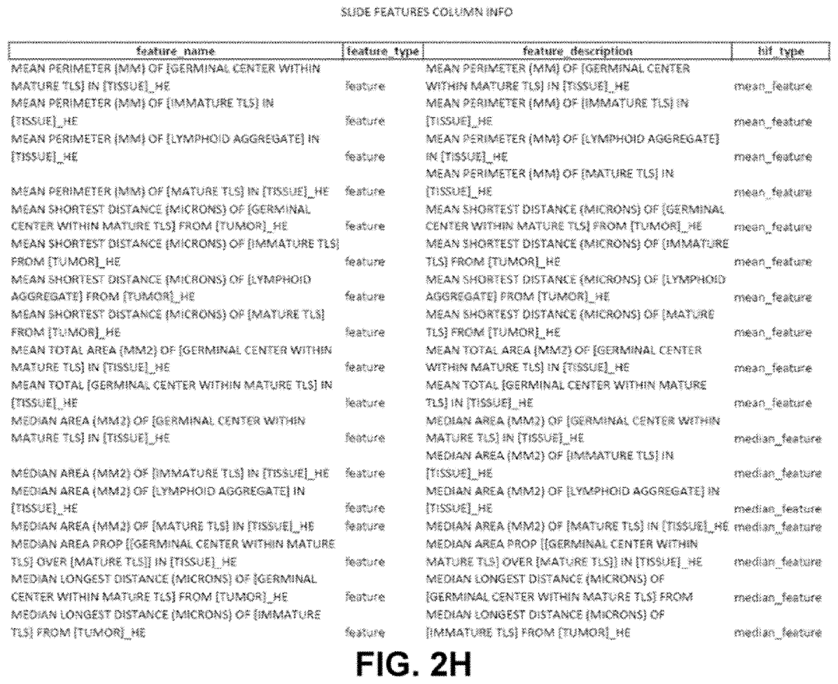

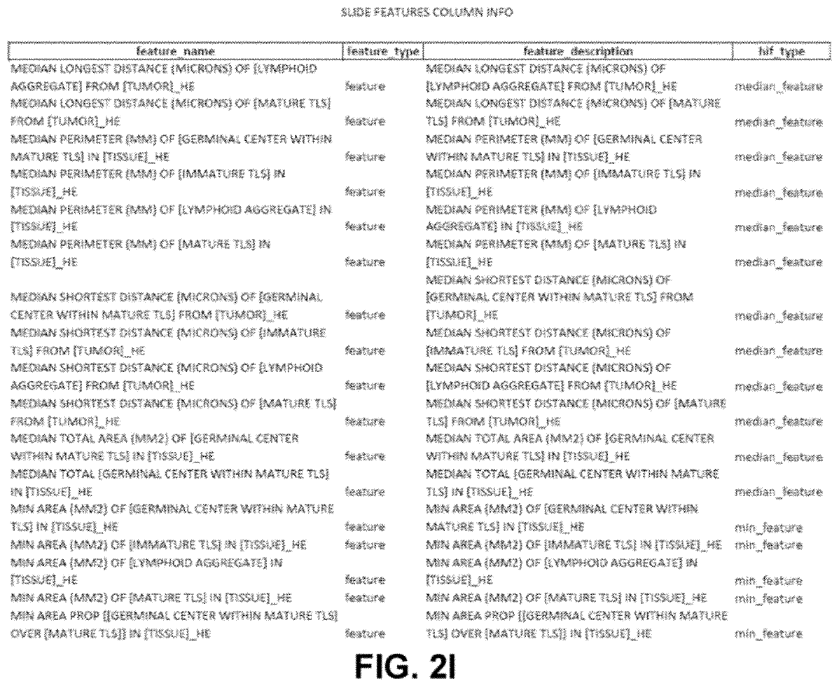

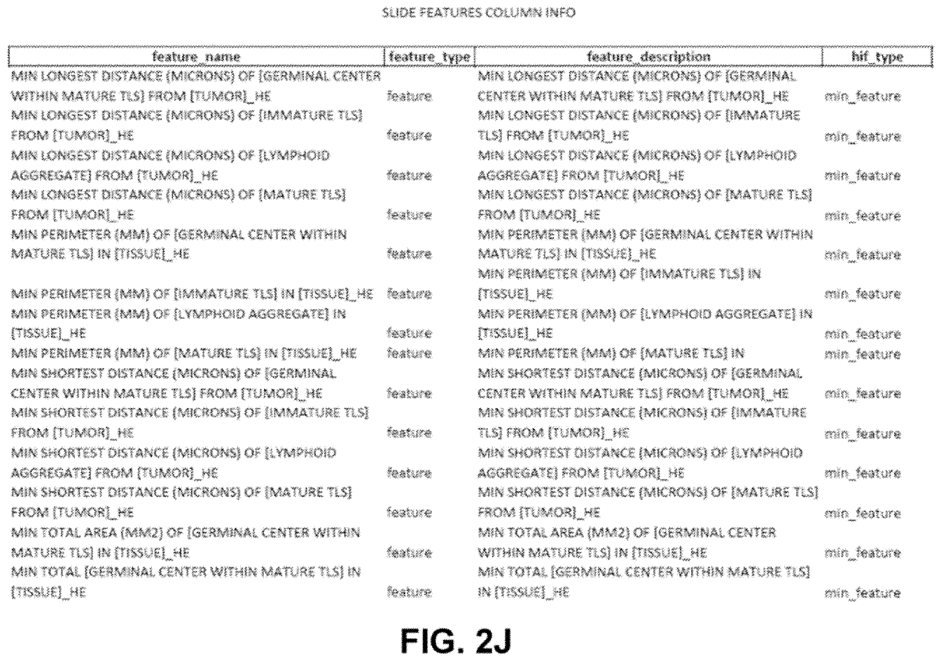

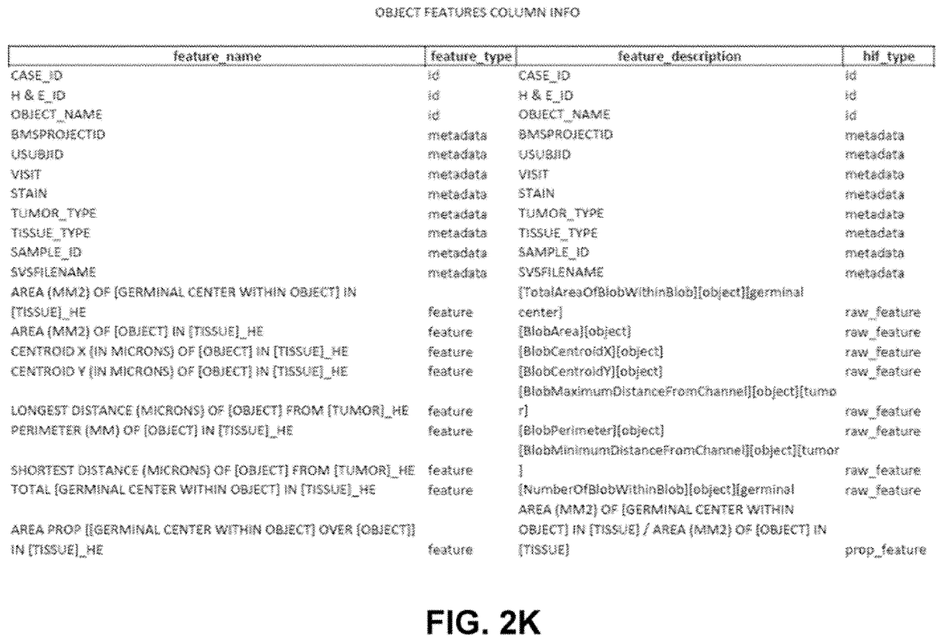

FIGS. 2A-2K show a plurality of tables that list TLS features 140 that may be extracted from by the TLS extractor 145. Each table includes a plurality of columns listing (1) a feature name, (2) a feature type that identifies whether the feature is an identification, metadata, or a feature, (3) a feature description that describes the extracted feature, and a human interpretable feature (HIF) type that indicates whether the feature is an identification, metadata, a raw feature, a minimum feature, a maximum feature, a median feature, a mean feature, a prop feature, a sum feature, or the like.

Referring back to FIG. 1, the TLS classification model 350 may process the respective set of TLS features 140 to classify the corresponding TLS region 135 as one of the first TLS maturation state (TLS1), the second TLS maturation state (TLS2), or the third TLS maturation state (TLS3). The first maturation state may be associated with lymphoid aggregates, the second maturation state may be associated the respective cluster of lymphocyte cells having primary follicles without any germinal center, and the third maturation state may be associated with the respective cluster of lymphocyte cells having primary follicles and secondary cells with a germinal center. Given that TLS2 and TLS3 tend to have a round shape and are usually larger than TLS1 and that TLS3 has a unique germinal center with lower lymphocyte density, the aforementioned TLS features 140 of area, roundness, and skewness can be interpreted by the trained TLS classification model 350 to classify each TLS region 135 accurately. Each TLS region 135 classified by the TLS classification model may correspond to a prognostic biomarker. The TLS classification model 350 may output TLS states 312 indicating the maturation state of each TLS region 135 classified by the TLS classification model 350.

In some examples, the application 160 executes an image augmenter 360 configured to augment the input histology image 110 based on the TLS states 312 output from the TLS classification model 350 for the one or TLS regions 135 identified in the input histology image 110. Here, the image augmenter 360 may generate a respective pixel mask 112 that highlights at least a perimeter of each corresponding TLS region 135 based on the maturation state (e.g., TLS1, TLS2, or TLS3) of the corresponding TLS region 135. The image augmenter 360 may generate a first pixel mask 112 for TLS regions 135 classified as TLS1, a second pixel mask 112 different than the first pixel mask 112 for TLS regions 135 classified as TLS2, and a third pixel mask 112 different than the first and second pixel masks 112 for TLS regions 135 classified as TLS3. That is, different pixel masks 112 may be visually distinguishable from one another. In some examples, the different pixel masks 112 are associated with different colors. The image augmenter 360 generates an output image 110A that augments the input histology image 110 by overlaying the respective pixel mask 112 generated for each of the TLS regions 135 onto the input histology image 110. The pixel masks 112 may be overlain as graphical features that highlight at least a perimeter of each corresponding TLS region 135, thereby serving as a visual cue indicating the location and corresponding classification (e.g., TLS1, TLS2, or TLS3) of each TLS region 135 identified in the output image 110A. As will become apparent, the image augmenter 360 may apply one or more post-processing rules to generate the output image 110A. As described in the preceding paragraphs, the application 160 may provide the output image 110 as TLS details 190 to the client device 111 for display on the screen 114.

In addition to maturation states, the TLS classification model 350 and/or TLS feature extractor 145 may be further configured to output/extract topological information associated with the TLS regions 135 such as coordinates of the TLS regions 135 as well as their proximity to the tumor bed and location relative to the tumor and/or stroma a compartment. In this manner, the image augmenter 360 or an image generator may process the topological information and any combination of the input histology image, the TLS states 312, the TLS regions 135, and the TLS features to generate a topological or heat map as the output image 110A that visually depicts the topological information associated with the TLS regions 135 that may be of interest.

With continued reference to FIG. 1, the application 160 may further execute a TLS scorer 150 for computing an overall TLS score 152 for the patient based on the TLS features 140 and corresponding TLS maturation states 312 for all the TLS regions 135 identified in the input histology image 110. The TLS scorer 150 may determine a total area of the tumor region 115 (denoted as ‘areaₜᵤₘₒᵣ’). The TLS scorer 150 may further determine a respective individual TLS area for each of the three TLS maturation states. For instance, the TLS scorer 150 may determine a first TLS area (denoted as ‘area_{TLS1}’) based on a total area of TLS regions classified as the first maturation state, a second TLS area (denoted as ‘area_{TLS2}’) based on a total area of TLS regions classified as the second maturation state, and a third TLS area (denoted as ‘area_{TLS2}’) based on a total area of TLS regions classified as the second maturation state. In some examples, the TLS scorer 150 computes the overall TLS score 152 as a linear weighted sum of the individual TLS areas divided by the tumor area as follows.

TLS score=(w1×area_{TLS1}+w2×area_{TLS2}+w3×area_{TLS3}) (1)

where w1, w2, w3 are corresponding weights. The optimal corresponding weights may be selected by performing a Cox regression analysis of overall survival with each of the individual TLS areas. In one example, w1 is equal to 0.81, w2 is equal to 0.84, and w3 is equal to 1.0, suggesting that TLS regions classified as the third maturation state (e.g., mature TLS) play a most important role in antitumor immune response.

Notably, statistical analysis applied to the overall TLS score 152, as well as individual TLS scores indicated by the first, second, and third TLS areas, may be used to predict various prognostic values for the patient such as predicting survival outcomes including, but not limited to overall survival and progression-free survival. Overall survival may be defined as the time from diagnosis to death or the last follow-up. Progression-free survival may be defined as the time from diagnosis to disease progression, death, or the last follow-up. Univariate and multivariate analyses may be performed with a Cox proportional hazard model. Clinical and pathological variables, such as tumor stage and grade, may be included in the multivariate analysis. Kaplan-Meier analysis and the log-rank test may be used to evaluate patient stratification by risk group. The TLS scores may be further assessed in associated with tumor state or grade. Higher overall TLS scores are indicative of significantly improved overall survival and progression-free survival compared to lower overall TLS scores. Overall survival and progression-free survival is still better for patients with low overall TLS scores than those where no TLS regions are identified. As such, overall TLS scores may be used to predict prognostic outcomes in lieu of using tumor stage predictions and/or prognostic outcomes predicted using tumor stage/grade may be further refined by the overall TLS scores.

In some scenarios, the application 160 performs post processing to adjust the output image 110A based on any combination of the TLS features 140, the TLS score(s) 152, and the TLS states 312. In particular, the application 160 may apply the one or more post processing rules 362 to modify the pixel masks 112 by fixing small and naked germinal centers, fixing TLS regions 135 without germinal centers which were classified as the third maturation state (mature TLS), fixing mosaics to address predictions of multiple classes on a same structure due to confusion by the TLS classification model, applying object level masking to remove false positive predictions of TLS within cancer and necrosis tissue regions, and/or applying cut-offs.

Referring to FIG. 3A, in some implementations, an example TLS classification model training process 300a trains the TLS classification model 350 to learn how to predict TLS states for TLS regions identified in histology images. The training process 300a obtains a training dataset 305 that includes a plurality of training histology images 310, 310a-n. Each training histology image 310 may contain a tumor microenvironment and include manual annotations 312 from qualified pathologists. The manual annotations 312 may identify one or more TLS regions 312a in the training histology image 310, and for each TLS region 312a identified, a ground-truth TLS maturation state 312b indicating that the corresponding TLS region 312a includes the first TLS maturation state, the second TLS maturation state, or the third TLS maturation state. Each TLS region 312a annotated in the training histology image 310 is represented by a respective cluster of lymphocyte cells.

The training process 300a executes a TLS feature extraction module 320 that receives each training histology image 310 and extracts a respective set of training TLS features 140 for each TLS region 312a. That is, for each TLS region 312a annotated in the training histology image 310, the TLS feature extraction module 320 may extract, from the respective cluster of lymphocyte cells representing the TLS region 312a, the respective set of training TLS features 140. TLS feature extraction module 320 may include the pre-trained tumor extraction model 450 and the pre-trained cell classification model 550 to generate lymphocyte density maps. The feature extraction module 320 may also include any other component or combination of components executed by the application 160.

The training TLS features may include, without limitation, an area 140a of the TLS region, a roundness 140b (i.e., the ratio of the area of TLS region 312a multiplied by 4 pi to a square of a perimeter of the TLS region), and a skewness 140c of the density of the respective cluster of lymphocyte cells representing the TLS region 312a. Based on the respective set of training TLS features 140 extracted for each TLS region 312a, the training process 300a trains the TLS classification model 350 using a classification and regression trees (CART) algorithm 340 to learn how to predict the ground-truth TLS state 312b for each corresponding TLS region 312a. In some examples, the training process 300a trains the CART algorithm 340 using scikit-learn package from the Python programming language version 3.6.11 (Python Software Foundation) using default parameter settings (criterion=gini; splitter=best; min_samples_split=2). The maximum depth of trees was determined to be 4 using 5-fold cross validation in the training dataset 305. Given the relative importance of TLS3, class weights for TLS1, TLS2, and TLS3 may be empirically set to 1, 2, and 3, respectively, during training.

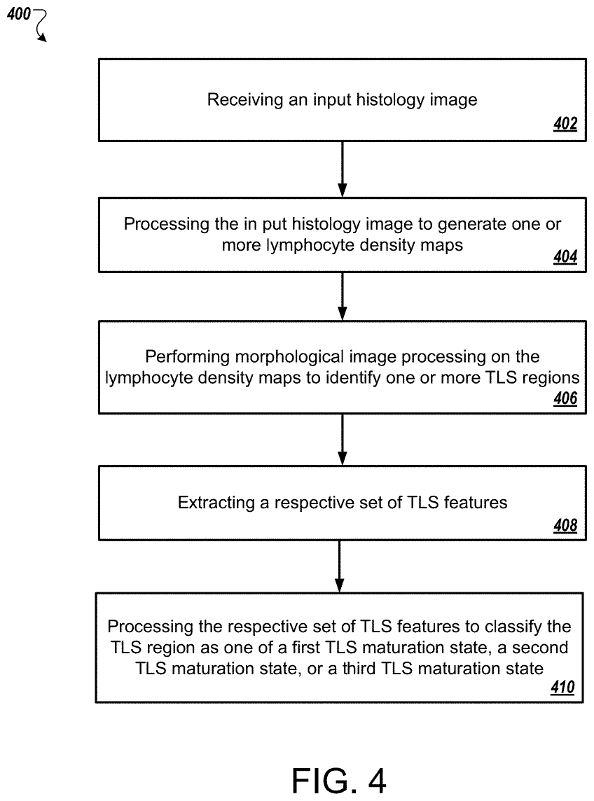

FIG. 4 is a flowchart of an example arrangement of operations for a method 400 of identifying, classifying, and quantifying TLS regions 135 within an input histology image 110. The method 400 may execute on the data processing hardware 142 of the remote system 141 and/or on the client device 111. At operation 402, the method 400 includes receiving the input histology image 110 for a patient diagnosed with cancer. The input histology image includes a plurality of image pixels. The input histology image 110 may include an H&E-stained image of a sample of the patient's tumor.

At operation 404, the method 400 includes processing, using a cell classification model 550, the input histology image 110 to generate one or more lymphocyte density maps 125 within the input histology image 110. At operation 406, the method 400 includes performing morphological image processing on the one or more lymphocyte density maps 125 to identify one or more TLS regions 135 within the input histology image 110. Here, each TLS region 135 is represented by a respective cluster of lymphocyte cells.

At operation 408, the method 400 includes, for each corresponding TLS region 135, extracting, from the respective cluster of lymphocyte cells representing the corresponding TLS region 135, a respective set of TLS features 140. At operation 410, the method 400 includes, for each corresponding TLS region 135, processing, using a TLS classification model 350, the respective set of TLS features to classify the corresponding TLS region as one of a first TLS maturation state, a second TLS maturation state, or a third TLS maturation state. The first TLS maturation state includes a lymphocyte aggregate of at least a threshold number of lymphocytes that do not contain high endothelial venules or germinal centers. The second TLS maturation state includes a dense aggregate of at least the threshold number of lymphocytes that contain high endothelial venules and do not contain any germinal centers. A third TLS maturation state includes a dense aggregate of at least the threshold number of lymphocytes that contain high endothelial venules and germinal centers.

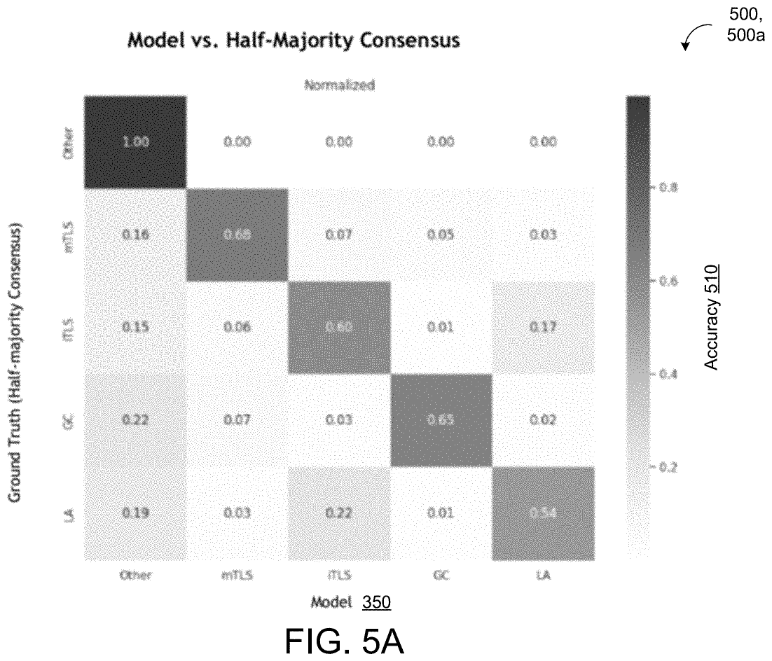

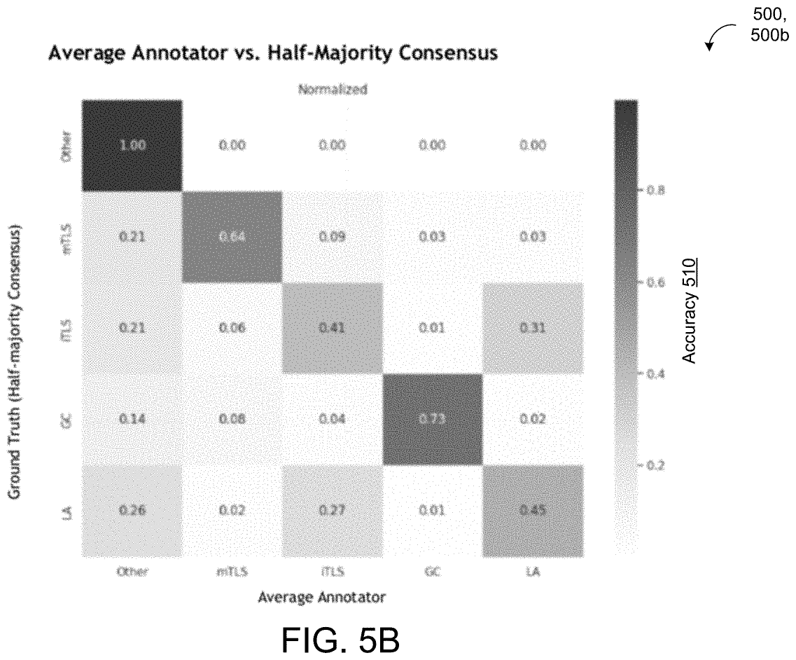

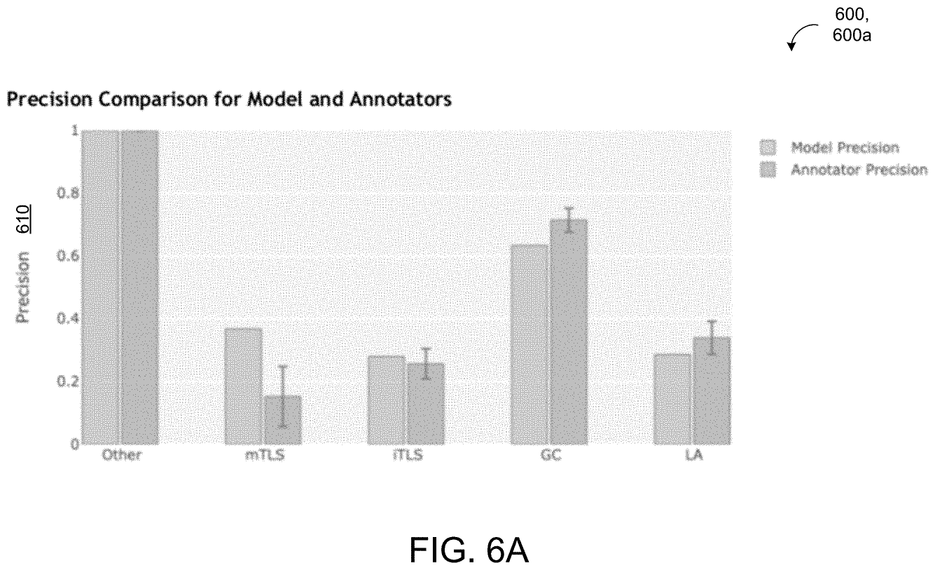

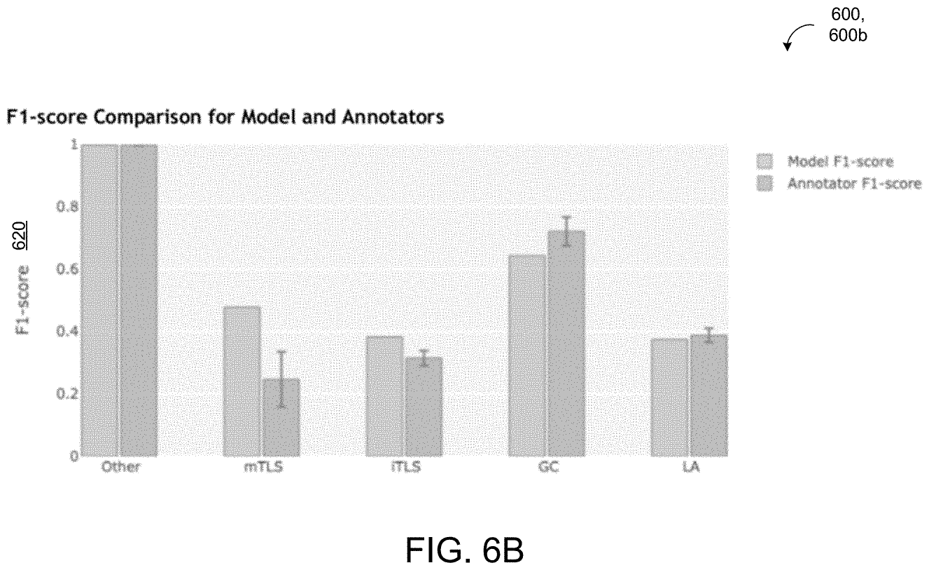

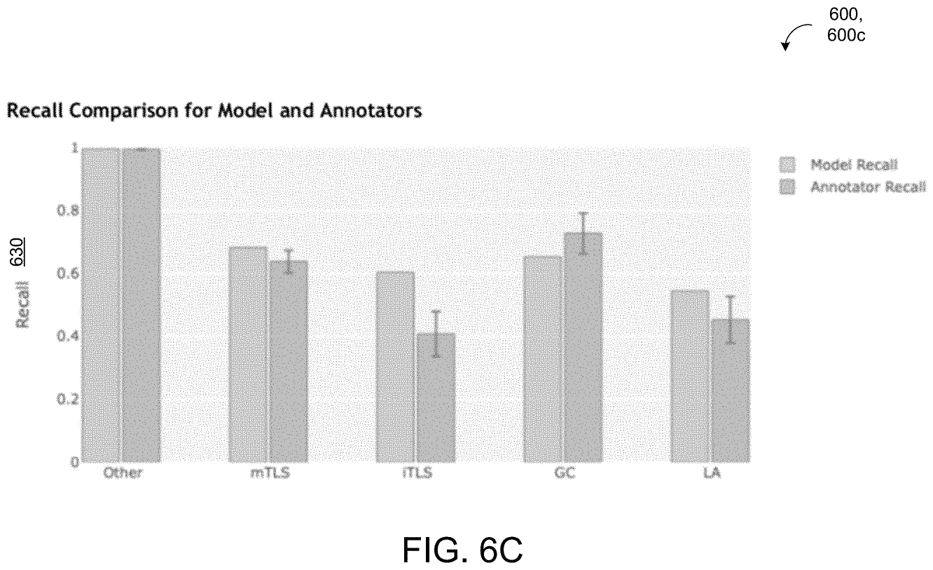

Advantageously, after training the TLS classification model 350, the accuracy of the TLS classification model 350 identifying and classifying TLS regions 135 within input histology images 110 is comparable (or in some scenarios even better) than the accuracy of pathologists classifying TLSs manually. For instance, confusion matrices 500 shown in FIGS. 5A and 5B depict confusion matrices 500 that compare accuracies 510 between pathologists and the trained TLS classification model 350. In particular, a first confusion matrix 500, 500a (FIG. 5A) shows normalized accuracies 510 of the TLS classification model 350 and a second confusion matrix 500, 500b (FIG. 5B) shows normalized accuracies of a pathologist annotator. Here, the confusion matrices 500 show accuracies 510 for each TLS maturation state 312 (e.g., mature TLS, immature TLS, germinal center, lymphoid aggregate, and other). The ground-truth maturation state for these input histology images 110 were generated by a majority consensus of 5 expert pathologists. Yet, FIGS. 6A-6C further illustrate plots 600 that compare TLS identification and classification performance between the TLS classification model 350 and pathologist annotators. In particular, a first plot 600, 600a (FIG. 6A) depicts a comparison of a precision score 610, a second plot 600, 600b (FIG. 6B) depicts a comparison of a F1-score 620, and a third plot 600, 600c (FIG. 6C) depicts a comparison of a recall score 630. Here, each respective plot 600 graphically represents the score for each of the different TLS maturations states 312.

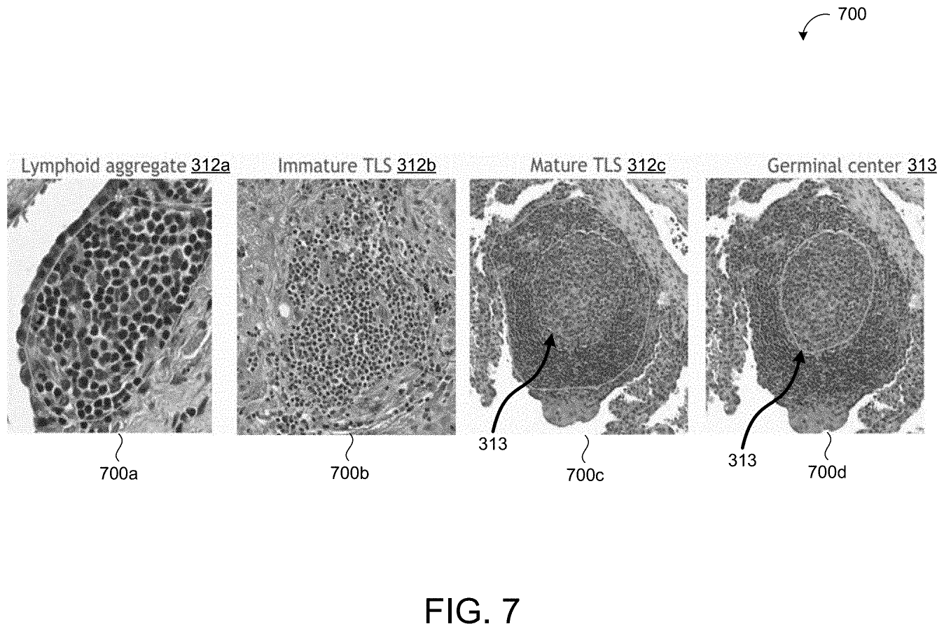

FIG. 7 depicts input histology images 700 each corresponding to a TLS maturation state 312 classified by the TLS classification model 350. Thus, the input histology images 110 (FIG. 1) may be interchangeable referred to as input histology images 700 with respect to FIG. 7. Notably, the input histology images 700 include a classified TLS maturation state 312, but are not annotated as an output image 110A. In some examples, the input histology images 700 correspond to an entire area of the input histology image 700. In other examples, the input histology images correspond only to the tumor regions 115 detected by the tumor detection model 450 or the TLS regions 135 identified by the morphological image processor 130 (FIG. 1) within the input histology image 700.

In the example shown, a first input histology image 700, 700a corresponds to a first TLS maturation state 312, 312a indicating a lymphoid aggregate maturation state. In particular, input histology images 700 corresponding to the first TLS maturation state 312a may include a dense aggregate of at least a threshold number of lymphocytes (e.g., 100 lymphocytes) that do not contain high endothelial venules nor germinal centers. A second input histology image 700, 700b corresponds to a second TLS maturation state 312, 312b indicating an immature TLS maturation state. Input histology images 700 corresponding to the second TLS maturation state 312b may include a dense aggregate of at least the threshold number of lymphocytes (e.g., 100 lymphocytes) that contain high endothelial venules (in contrast to the first TLS maturation state 312a) but do not contain any germinal centers. A third input histology image 700, 700c corresponds to a third TLS maturation state 312, 312c indicating a mature TLS maturation state. Input histology images 700 corresponding to the third TLS maturation state 312c may include the dense aggregate of at least the threshold number of lymphocytes (e.g., 100 lymphocytes) that contain high endothelial venules and germinal centers 313 (in contrast to the first and second TLS maturation states 312a, 312b).

With continued reference to FIG. 7, a fourth input histology image 700, 700d illustrates a germinal center 313. In some implementations, germinal centers 313 are not a distinct TLS maturation state 312, but rather the germinal centers 313 are a feature of the mature TLS maturation state 312c. In other implementations, the TLS classification model 350 classifies germinal centers 313 as distinct TLS maturation state 312 independent from the other TLS maturation states 312. Input histology images 700 with germinal centers 313 include a paler, less dense region at a center of mature TLSs (e.g., third TLS maturation state 312c) surrounded by dense lymphocyte regions. Although not depicted in FIG. 7, the TLS classification model 350 may also classify a fourth TLS maturation state (not shown) indicating a non-TLS region (e.g., zero TLS region present in the input histology image 700) or other region. As used herein the first TLS maturation state 312a, the second TLS maturation state 312b, and the third TLS maturation state 312c may interchangeably be referred to as lymphoid aggregate TLS maturation state 312a, immature TLS maturation state 312b, and mature TLS maturation state 312c, respectively.

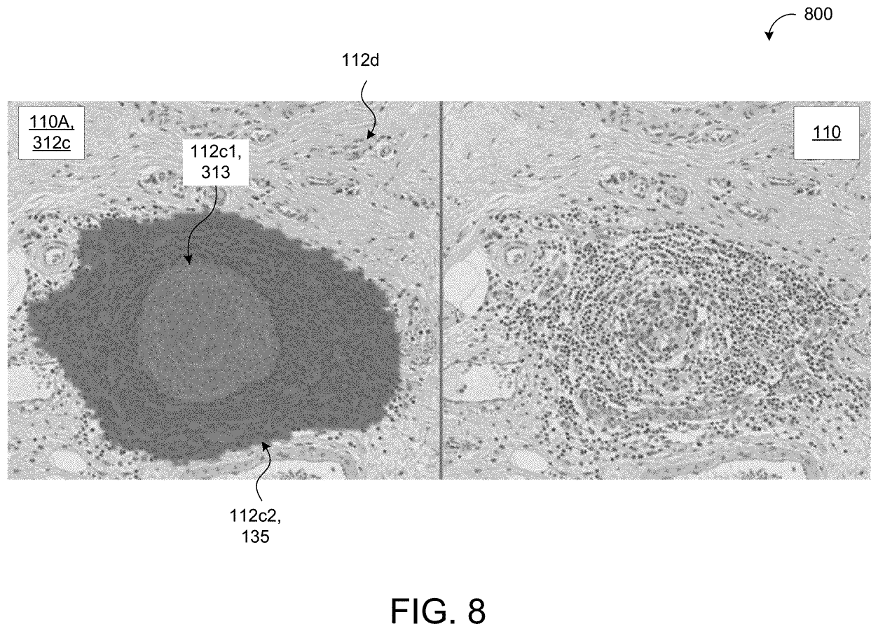

FIGS. 8-10 depict exemplary input histology images 110 and the corresponding output images (e.g., TLS augmented histology images) 110A generated by the image augmenter 360 (FIG. 1). Stated differently, the application 160 receives, as input, the exemplary input histology images 110 (right) shown in FIGS. 8-10, as input, and generates, as output, the output images 110A (left). In some examples, the image augmenter 360 generates a respective pixel mask 112 that highlights at least a perimeter of the corresponding TLS region 135. In other examples, the respective pixel mask 112 highlights an entire area of the corresponding TLS region 135. Thereafter, the image augmenter 360 may generate the output image 110A that augments the input histology image 110 by overlaying the respective pixel mask 112 generated for each of the TLS regions 135 onto the input histology image 110.

Moreover, the image augmenter 360 generates a first pixel mask 112, 112a for each corresponding TLS region 135 classified as the first TLS maturation state 312a, a second pixel mask 112, 112b for each corresponding TLS region 135 classified as the second maturation state 312b, and a third pixel mask 112, 112c for each corresponding TLS region 135 classified as the third maturation state 312c. Notably, each pixel mask 112 is visually distinguishable from the other pixel masks 112 such that the output image 110A visually depicts the different maturation states 312 using the visually distinct pixel masks 112. As such, the output images 110A be displayed on the screen 114 of the user device 111 such that the user 10 (FIG. 1) may easily visualize the different TLS maturation states 312 included in the output images 110A. Optionally, the image augmenter 360 may generate a fourth pixel mask 112, 112d for each corresponding TLS region 135 classified as the non-TLS region.

For example, FIG. 8 shows a graphical representation 800 of an input histology image 110 (right) representing tissue of the mature TLS maturation state 312c and a corresponding output image 110A (left) that includes the third pixel mask 112c that highlights the area of the TLS region 135 classified as the mature TLS maturation state 312c. Yet, the mature TLS maturation state 312c includes a germinal center 313 encompassed by the TLS region 135 corresponding to the mature TLS maturation state 312c. To that end, the third pixel mask 112c includes an inner third pixel mask 112c1 that highlights the area of the germinal center 313 and an outer third pixel mask 112c2 that highlights the area of the mature TLS maturation state 312c.

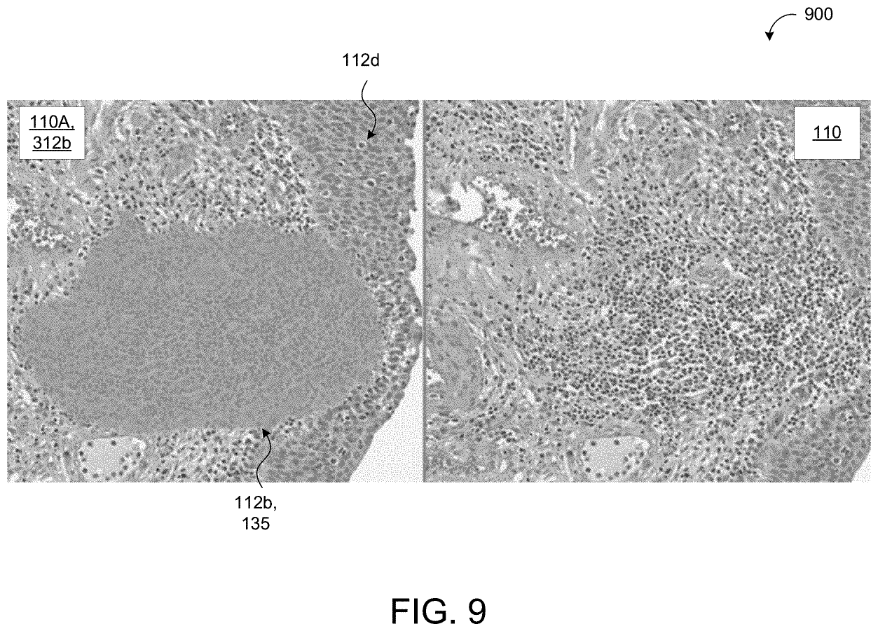

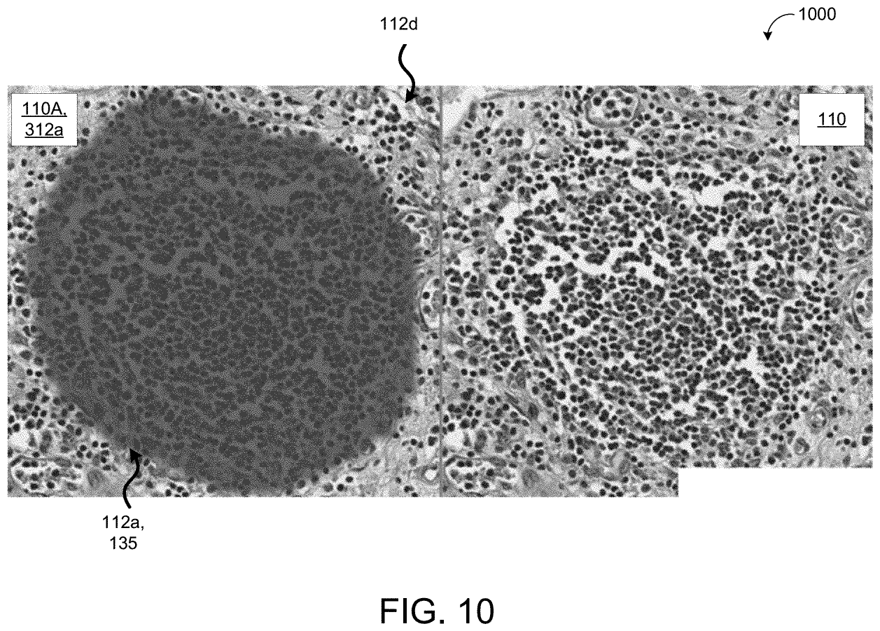

FIG. 9 illustrates a graphical representation 900 of an input histology image 110 (right) representing tissue of the immature TLS maturation state 312b and a corresponding output image 110A (left) that includes the second pixel mask 112b that highlights the area of the TLS region 135 classified as the immature TLS maturation state 312b. In yet another example, FIG. 10 illustrates a graphical representation 1000 of an input histology image 110 (right) representing tissue of the lymphoid aggregate TLS maturation state 312a and a corresponding output image 110A (left) that includes the first pixel mask 112a that highlights the area of the TLS region 135 classified as the lymphoid aggregate TLS maturation state 312a. Moreover, the output images 110A shown in each of the graphical representations 800, 900, 1000 further include a fourth pixel mask 112d that highlights the area of the output image 110A corresponding to the non-TLS maturation TLS region (e.g., non-TLS maturation state).

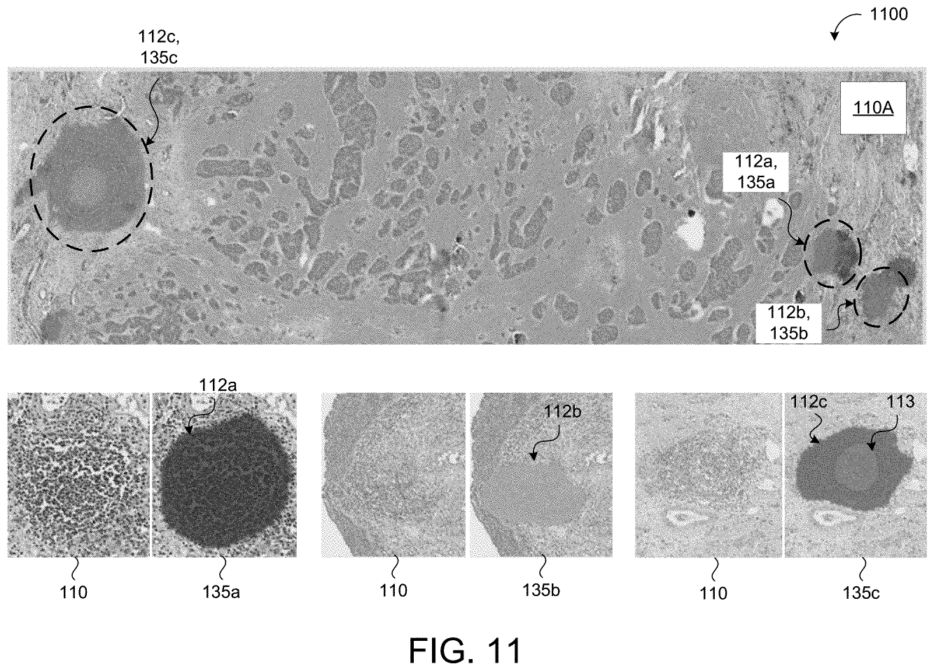

Referring now to FIG. 11, in some implementations, an input histology image 110 includes several classified TLS maturation states 312. For example, a graphical representation 1100 shows an output image 110A that includes three TLS regions 135 corresponding to each of the first, second, and third TLS maturation states 312a-c. Here, each respective pixel mask 112 overlain on the input histology image readily indicates to the user the different identified TLS regions 135 and the corresponding classified TLS maturation states 312. As shown in FIG. 11, the output image 110A includes a first TLS region 135, 135a classified as the lymphoid aggregate TLS maturation state 312a, a second TLS region 135, 135b classified as the immature TLS maturation state 312b, and a third TLS region 135, 135c classified as the mature TLS classification state 312c including the germinal center 313. Moreover, below the output image 110A, expanded views of the identified TLS regions 135 are shown adjacent to the corresponding input histology image 110. For instance, the first TLS region 135a includes the first pixel mask 112a highlighting the area of the first TLS region 135a as the lymphoid aggregate TLS maturation state 312a, the second TLS region 135b includes the second pixel mask 112b highlighting the area of the second TLS region 135b as the immature TLS maturation state 312b, and the third TLS region 135c includes the third pixel mask 112c highlighting the area of the third TLS region 135c as the mature TLS maturation state 312c. Next to each expanded TLS region 135, is the corresponding portion of the input histology image 110 input to the application 160 that corresponds to the TLS region 135.

Referring back to FIG. 1, in some implementations, the image augmenter 360 applies one or more post-processing rules 362 before generating the output image 110A. That is, in some scenarios, the TLS classification model 350 classifies a TLS region 135 as a particular TLS maturation state 312 that does not satisfy a threshold (e.g., post-processing threshold). Thus, applying the post-processing rules 362 filters out classified TLS maturation states 312 that fail to satisfy the one or more post-processing rules. As such, the image augmenter 360 applies the post-processing rules 362 to correct any false-positive or otherwise incorrect classifications generated by the TLS classification model 350. For instance, the post-processing rules 362 may include, but are not limited to, fixing small and naked germinal centers, fixing a mature TLS without germinal centers, fixing mosaics to address predictions of multiple classes on the same structure due to model confusion, object level masking to remove false positive predictions of a TLS within cancer and necrosis tissue regions, and/or applying cut-offs.

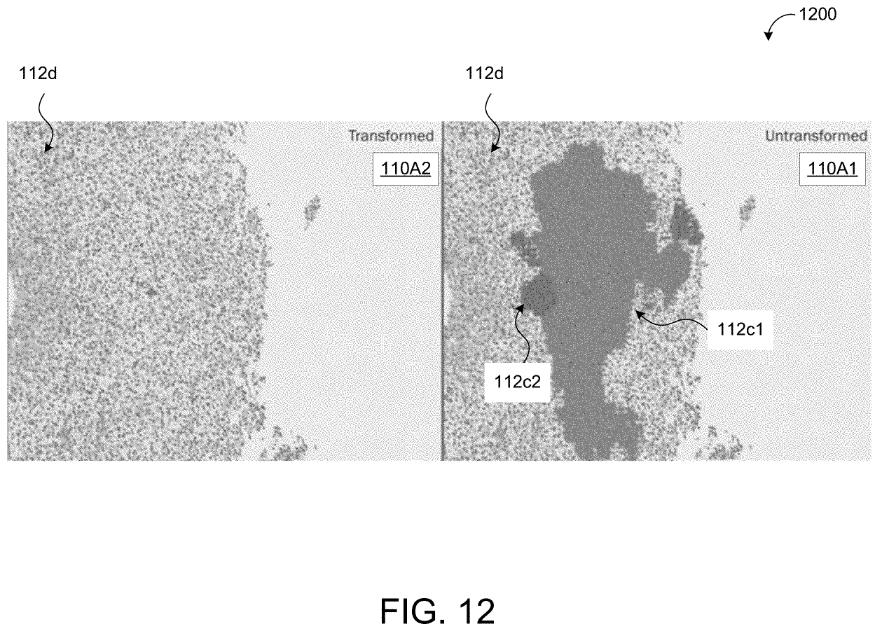

FIGS. 12-15 illustrate output images 110A generated by the image augmenter 360 both applying and not applying post-processing rules 362. When the image augmenter 360 does not apply the post-processing rules 362, the output images 110A may be referred to as untransformed output images 110A, 110A1. On the other hand, when the image augmenter 360 applies the post-processing rules 362, the output images 110A may be referred to as transformed output images 110A, 110A2. For example, FIG. 12 illustrates a graphical representation 1200 of output images 110A when applying a post-processing rule 362 to fix (i.e., filter) small and naked germinal centers. As shown in FIG. 12, an untransformed output image 110A1 includes a germinal center 313 partially surrounded by TLS regions 135 classified as the mature TLS maturation state 312c and the non-TLS maturation state denoted by their respective pixel masks 112. Here, a post-processing rule 362 defines that for germinal centers 313 that fail to satisfy a threshold amount of TLS region 135 classified as mature TLS maturation state 312c surrounding the germinal center 313 (e.g., 70% of the germinal center 313 surrounded by mature TLS), the image augmenter 360 re-classifies the germinal center 313 as the TLS maturation state 312 that surrounds a majority of the germinal center 313.

For example, as shown in FIG. 12, an untransformed output image 110A1 includes the outer third pixel mask 212c2 (e.g., indicating mature TLS maturation state 312c) only partially surrounding the inner third pixel mask 212c1 (e.g., indicating germinal center 313) thereby failing to satisfy the threshold amount. Thus, the image augmenter 360 re-classifies the germinal center 313 as the non-TLS maturation state because a majority of the perimeter of the germinal center 313 is surrounded by non-TLS regions. As a result, a transformed output image 110A2 removes (i.e., filters) the germinal center 313 such that the transformed output image 110A2 only includes the fourth pixel mask 112d. Alternatively, the post-processing rule 362 may define that for germinal centers 313 that have an area that fails satisfy a threshold area (e.g., 4480 μm2), the image augmenter 360 re-classifies the germinal center 313 as the TLS maturation state 312 that surrounds a majority of perimeter of the germinal center 313.

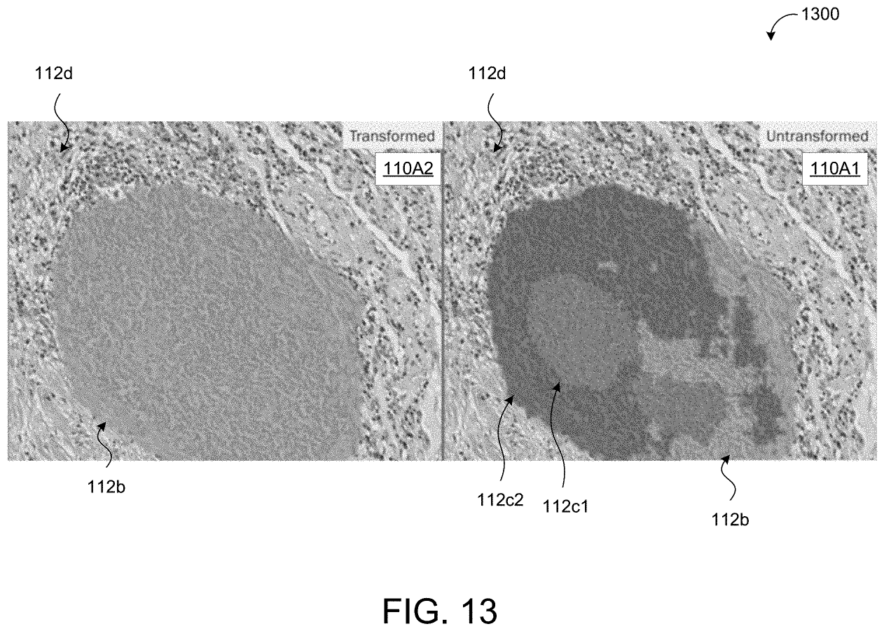

Referring now to FIG. 13, in some implementations, the post-processing rules 362 are configured to fix classified mature TLS maturation states 312c without germinal centers 313. Here, the image augmenter 360 re-classifies TLS regions classified as mature TLS maturation states 312c that are not connected to a germinal center 313 as the immature TLS maturation state 312b. For instance, the mature TLS maturation state 312c regions may need to fully encompass the germinal center 313 or partially encompass the germinal center 313 satisfying a threshold value. As shown in FIG. 13, a graphical representation 1300 includes an untransformed output image 110A1 includes a second pixel mask 112b (e.g., indicating immature TLS maturation state 312b), an inner third pixel mask 112c1 (e.g., indicating the germinal center 313), an outer third pixel mask 112c2 (e.g., indicating the mature TLS maturation state 312c). In this example, the outer third pixel mask 112c2 fails to encompass the germinal center 313 by the threshold value. That is, the outer third pixel mask 112c2 only partially encompasses the germinal center 313 but not enough to satisfy the threshold value. Thus, in this scenario, the image augmenter 360 re-classifies the mature TLS maturation state 312c and the germinal center 313 as the immature TLS maturation state 312b as shown in a transformed output image 110A2 with the second pixel mask 112b. The output images 110A also include the fourth pixel mask 112d corresponding to the non-TLS regions of the output image 110.

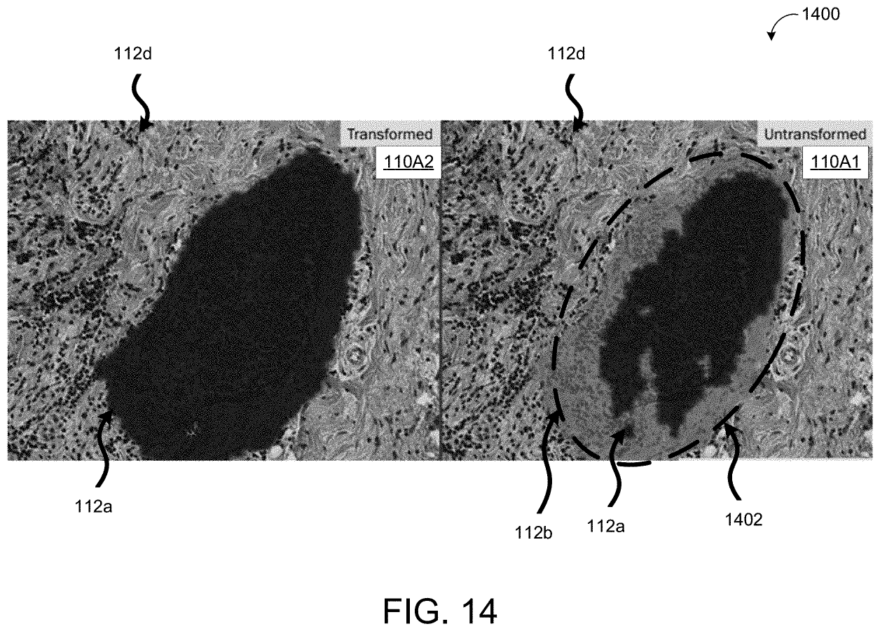

Referring now to FIG. 14, in some examples, the post-processing rules 362 are configured to fix mosaics 1402 included in the output image 110A. Here, mosaics 1402 refer to a single TLS region 135 that includes multiple classified TLS maturation states 312. In some configurations, when the mosaic 1402 includes at least the immature TLS maturation state 312b and the lymphoid aggregate TLS maturation state 312a, the image augmenter 360 re-classifies the entire mosaic 1402 as the immature TLS maturation state 312b based on determining that the mosaic 1402 includes a threshold ratio (e.g., 70 percent) of the immature TLS maturation state 312b. Otherwise, the image augmenter 360 re-classifies the entire mosaic 1402 as the lymphoid aggregate TLS maturation state 312a. In other configurations, where the mosaic 1402 includes at least the immature TLS maturation state 312b and the mature TLS maturation state 312c, the image augmenter 360 re-classifies the entire mosaic 1402 as the mature TLS maturation state 312c based on determining that the mosaic includes a threshold ratio (e.g., 70 percent) of the mature TLS maturation state 312c. Otherwise, the image augmenter 360 re-classifies the entire mosaic 1402 as the immature TLS maturation state 312b. In yet other configurations, where the mosaic 1402 includes at least the lymphoid aggregate TLS maturation state 312a and the mature TLS maturation state 312c, the image augmenter 360 re-classifies the entire mosaic 1402 as the mature TLS maturation state 312c based on determining that the mosaic 1402 includes a threshold ratio (e.g., 70 percent) of the mature TLS maturation state 312c. Otherwise, the image augmenter 360 re-classifies the entire mosaic 1402 as the lymphoid aggregate TLS maturation state 312a.

As shown in FIG. 14, a graphical representation 1400 includes an untransformed output image 110A1 depicting a mosaic 1402 that includes the first pixel mask 112a (e.g., indicating the lymphoid aggregate TLS maturation state 312a) and the second pixel mask 112b (e.g., indicating the immature TLS maturation state 312b). Here, the mosaic 1402 does not satisfy the threshold ratio of the immature TLS maturation state 312b. As such, the image augmenter 360 re-classifies the entire area of the mosaic 1402 as the lymphoid aggregate maturation state 312a as shown in transformed output 110A2 that includes the first pixel mask 112a. Notably, the transformed output 110A2 eliminates the mosaic 1402 because the TLS region only includes a single TLS maturation state 312. The output images 110A also include the fourth pixel mask 112d corresponding to the non-TLS regions of the output image 110.

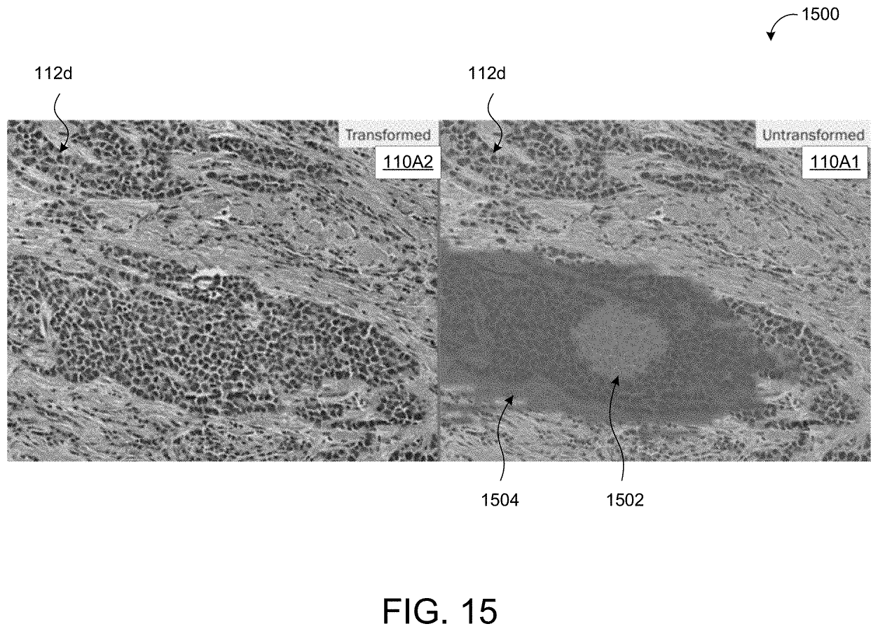

Referring now to FIG. 15, in some implementations, the post-processing rules 362 are configured to remove false positive predictions of TLS maturations states 312 within cancerous and necrosis tissue regions. In particular, the image augmenter 360 determines whether a proportion of cancer and necrosis in an object or TLS region classified as the first, second, or third TLS maturations state 312a, 312b, 312c, satisfies a threshold ratio (e.g., 20 percent) of the object or TLS region. In response to determining that the proportion of cancer and necrosis satisfies the threshold ratio, the image augmenter 360 re-classifies the TLS maturation state 312 as the non-TLS maturation state. For example, as shown in FIG. 15, a graphical representation 1500 includes an untransformed output image 110A1 that includes a cancer pixel mask 1502 and a necrosis pixel mask 1504. In this example, the cancer pixel mask 1502 and the necrosis pixel mask 1504 satisfy the threshold ratio of the tissue, and thus, the image augmenter 360 re-classifies the cancer pixel mask 1502 and the necrosis pixel mask 1504 as the non-TLS maturations state 312d. Thus, transformed output image 110A2 includes only the fourth pixel mask 112d corresponding to the non-TLS region of the transformed output image 110A.

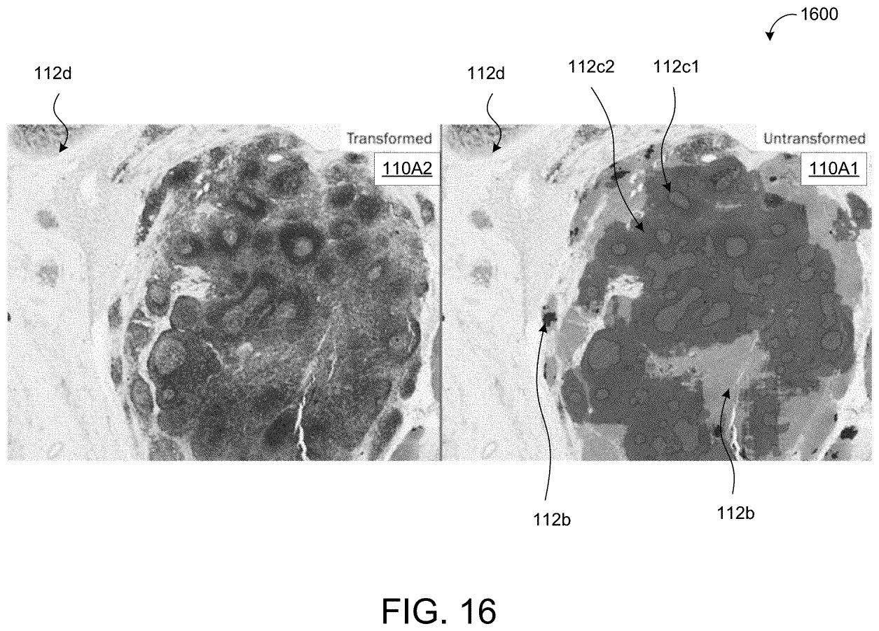

Referring now to FIG. 16, in some examples, the post-processing rules 362 are configured to apply cut-offs that filter classified TLS maturation states 312 that fail to satisfy either a minimum threshold area, a maximum threshold area, and/or a maximum number of germinal centers 313. TLS maturation states 312 that fail to satisfy the thresholds are re-classified as non-TLS regions. In particular, the lymphoid aggregate TLS maturation state 312a may have minimum threshold area (e.g., 0.0008 mm\hat{ }2) and no maximum threshold area. On the other hand, the immature TLS maturation state 312b may include a minimum threshold area (e.g., 0.018 mm\hat{ }2) and a maximum threshold area (e.g., 2.0 mm\hat{ }2). Moreover, the mature TLS maturation state 312c may have a maximum threshold number of germinal centers 313 (e.g., 8 germinal centers 313). For instance, if the mature TLS maturation state 312c includes a number of germinal centers 313 that exceeds the maximum threshold number, the image augmenter 360 re-classifies the mature TLS maturation state 312c as the non-TLS region. As shown in FIG. 16, a graphical representation 1600 includes an untransformed output image 110A1 that includes the first pixel mask 112a, the second pixel mask 112b, the inner third pixel mask 112c1, the outer third pixel mask 112c2, and the fourth pixel mask 112d. Yet, none of the pixel masks 112 satisfy the cut-off thresholds, and thus, the image augmenter 360 re-classifies each of the first, second, and third TLS maturation states 312a-c as the fourth TLS classification state 312d as shown in transformed output image 110A2. That is, the transformed output image 110A2 only includes the fourth TLS classification state 312d.

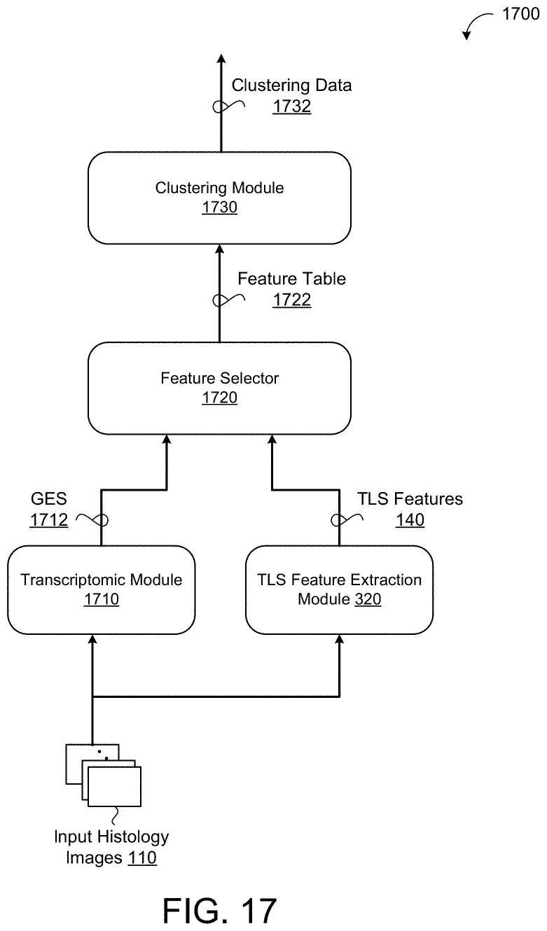

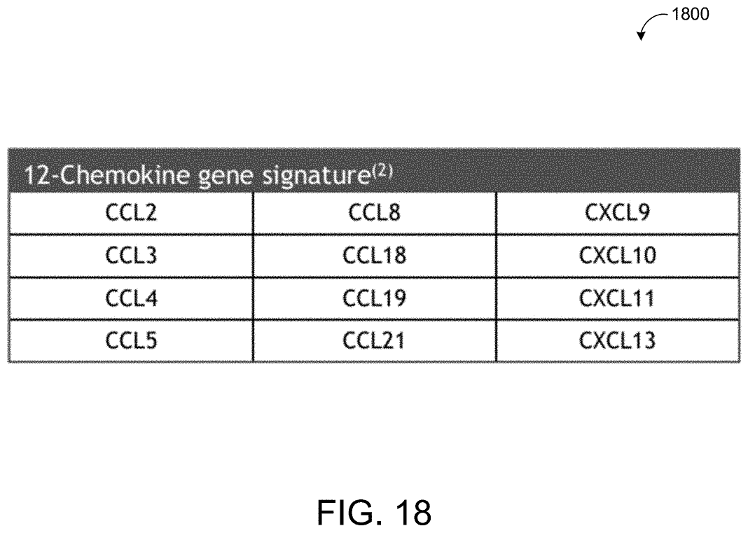

FIG. 17 illustrates a process flow diagram 1700 for validating extracted TLS features 140 using ribonucleic acid (RNA) sequence analysis or transcriptomic analysis correlation. That is, various gene signatures of TLSs have been studied that are related to either chemokines or cell populations. For example, FIG. 18 shows a table 1800 of a 12-chemokine gene signature derived by correlating a metagene related to inflammation and associated with enhanced patient survival in colorectal cancer, melanoma, and breast cancer. The 12-chemokine gene signature of table 1800 includes CCL2, CCL3, CCL4, CCL5, CCL8, CCL18, CCL19, CCL21, CXCL9, CXCL10, CXCL11, and CXCL13. In another example, an 8-gene signature representing T follicular helper (TFH) cells, which in particular includes CXCL13, characterizes breast cancer. In yet another example, a 19-gene signature related to T helper type 1 (Th1) cells and B cells indicate the presence of TLS. Despite the various gene signatures that correlate to TLS presence, recently there have been a limited number of studies investigating the most accurate TLS gene signature.

Referring back to FIG. 17, while TLS detection by immunohistochemistry in tissue sections is a robust and specific approach, the process flow diagram 1700 aims to compare several gene signatures extracted from TLS-positive cancer tissue. As will become apparent, a heterogeneity of gene expression among different cancer types leads to a better understanding of gene signatures that correlate to TLS presence. In particular, the process flow diagram 1700 includes the TLS feature extraction module 320, a transcriptomic module 1710, a feature selector 1720, and a clustering module 1730. The TLS feature extraction module 320 is configured to receive, as input, the input histology images 110 and extract TLS features 140 corresponding to each respective input histology image 110. For example, the TLS feature extraction module 320 may extract the TLS features 140 using the TLS feature extractor 145 (FIG. 1).

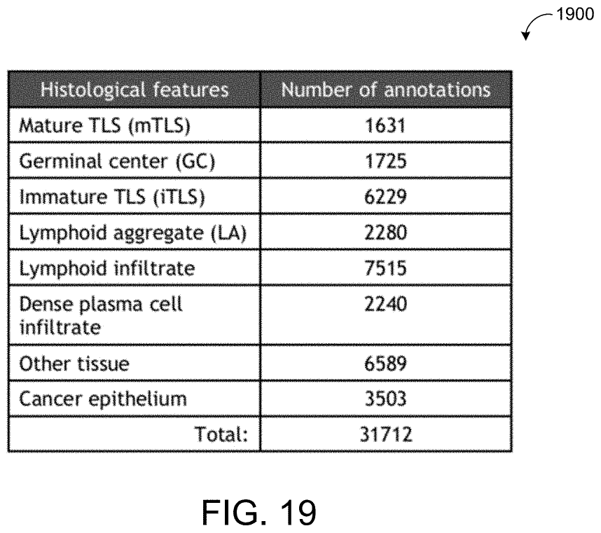

The transcriptomic module 1710 is configured to receive, as input, the input histology images 110 and generate, as output, a gene expression signature (GES) 1712 for each respective input histology image 110. Here, the transcriptomic module 1710 may generate the GES 1712 by extracting the RNA-sequence from the respective input histology image 110. Using the TLS features 140 and the GES 1712 generated for each of the input histology images 110, the feature selector 1720 generates a feature table 1722. That is, for each respective input histology image 110, the feature extractor 1720 pairs the TLS features 140 and the GESs 172 derived from the respective input histology image 110 in the feature table 1722. The feature table 1722 includes the pairings for all of the received input histology images 110. As such, the feature table 1722 structures the TLS features 140 and the GESs 1712 such that the clustering module 1730 may determine correlations between the TLS features and the GESs 1712. In some examples, the feature table 1722 includes other TLS features 140 and the corresponding number of annotations for each TLS feature 140 in the set of input histology images as shown in table 1900 (FIG. 19). In some implementations, the feature selector 1720 may filter to the feature table 1722 to only include particular TLS features 140. For example, the feature selector 1720 may apply a linear regression lasso penalty to generate the feature table 1722.

With continued reference to FIG. 17, the clustering module 1730 is configured to receive, as input, the clustering table 1722 and generate, as output, the correlation data 1732. Notably, using gene signature data that indicates the presence and classification of TLSs, the clustering module 1730 may validate that the extracted TLS features 140 correlate to the presence and classification of TLSs in input histology images 110. Moreover, the clustering module 1730 may leverage the extracted TLS features 140 to further determine gene signatures that indicate the presence and classification of TLSs in tissue. That is, the clustering module 1730 may further determine gene signatures that can identify TLSs that are not yet known.

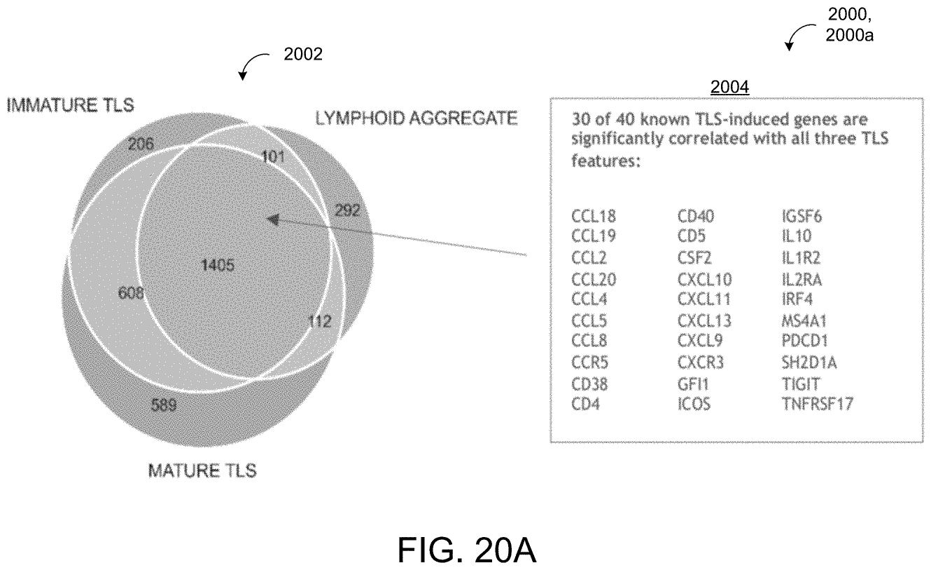

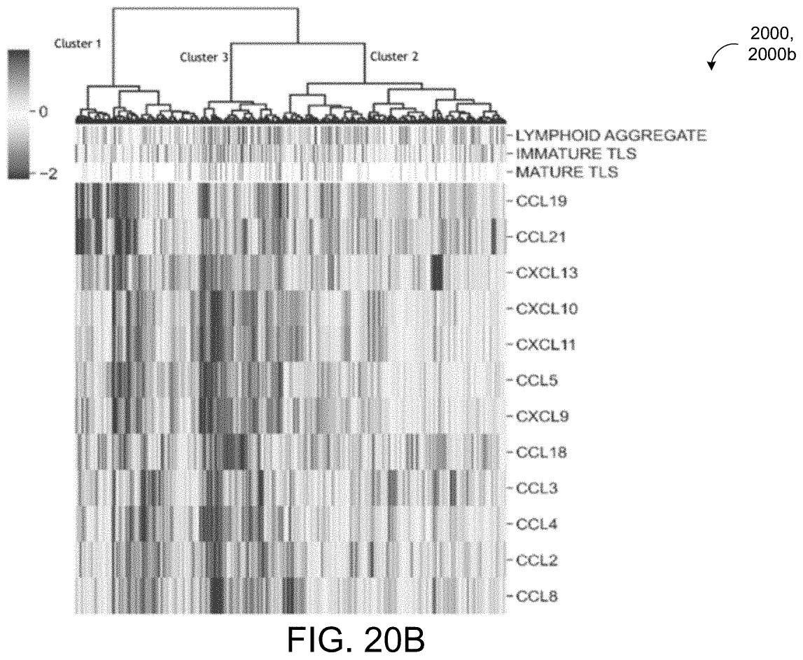

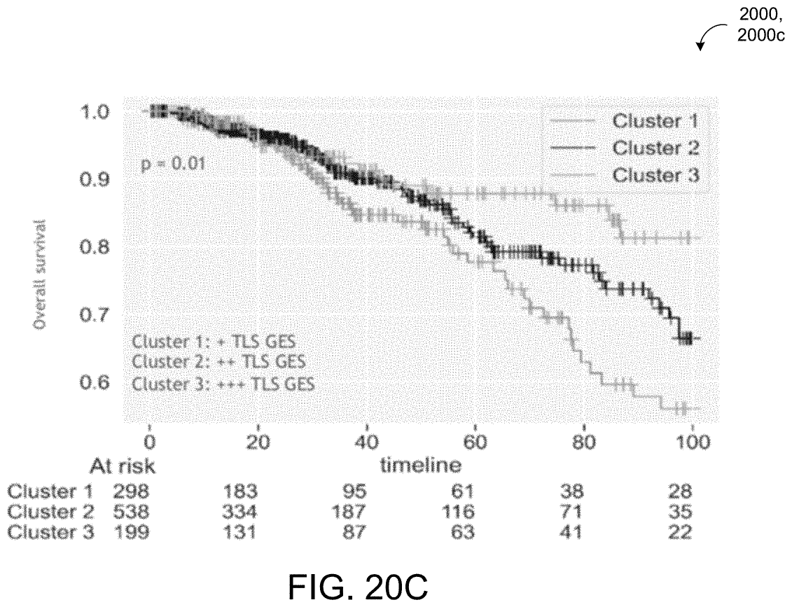

For instance, FIGS. 20A-20C show graphical representations 200 of example correlation data 1732 (FIG. 17) validating that the TLS features 140 strongly correlate to GESs in an example breast cancer gene (BRCA) analysis. In particular, graphical representation 2000a (FIG. 20A) illustrates correlation diagram 2002 showing that the TLS maturation states 312 and TLS features 140 correspond to TLS-induced genes shown in table 2004 in the BRCA analysis. Moreover, the correlation diagram 2002 depicts the TLS-induced genes that occur in each of the first, second, and third TLS maturation states 312a-c and the TLS-induced genes the correlate to individual TLS maturation states 312. Further processing of the correlation diagram 2002 by the clustering module 1730 (FIG. 17) may generate signatures for certain cancers. Simply put, the correlation diagram 2002 highlights that the TLS features 140 strongly correlate GESs for input histology images 110 of the BRCA.

FIG. 20B illustrates a graphical representation 2000b of a hierarchical clustering plot. Here, the plot includes cluster 1 corresponding to low expression breast cancer samples, cluster 2 corresponding to intermediate expression breast cancer samples, and cluster 3 corresponding to high expression breast cancer samples. Along the y-axis, the first, second, and third TLS maturation states 312a-c and TLS-induced genes are plotted for each of the breast sample clusters. FIG. 20C illustrates a graphical representation 2000c of a plot depicting an x-axis as a timeline in months and a y-axis as an overall survival rate of the patients from the breast cancer samples. Thus, the graphical representations 2000c shows that breast cancer samples in clusters with up-regulated chemokines have a higher long-term overall survival rate.

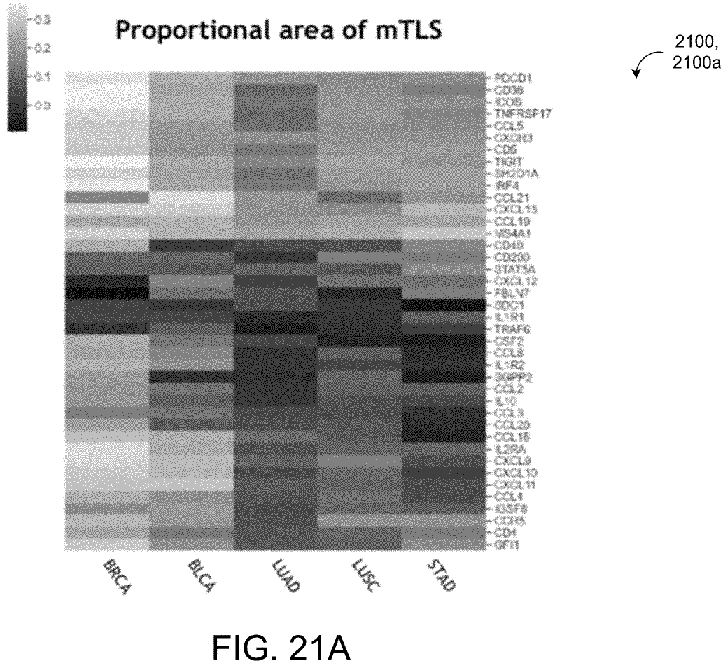

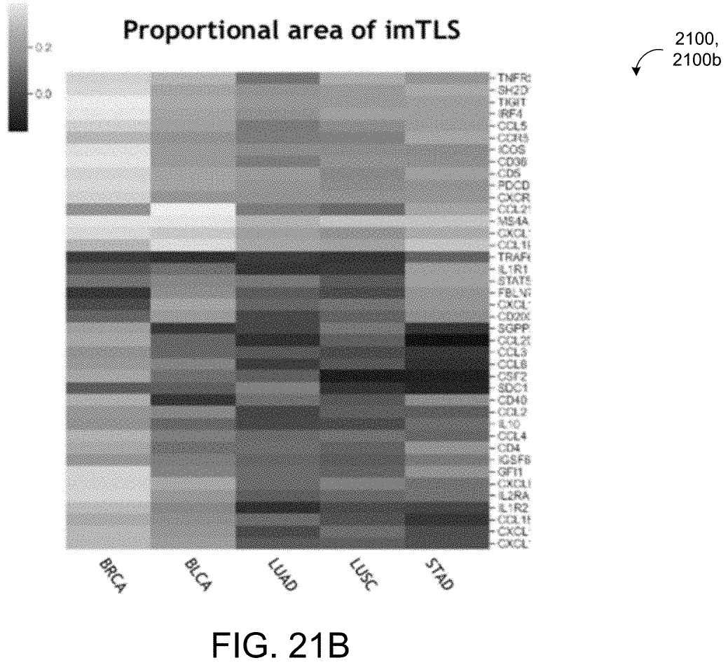

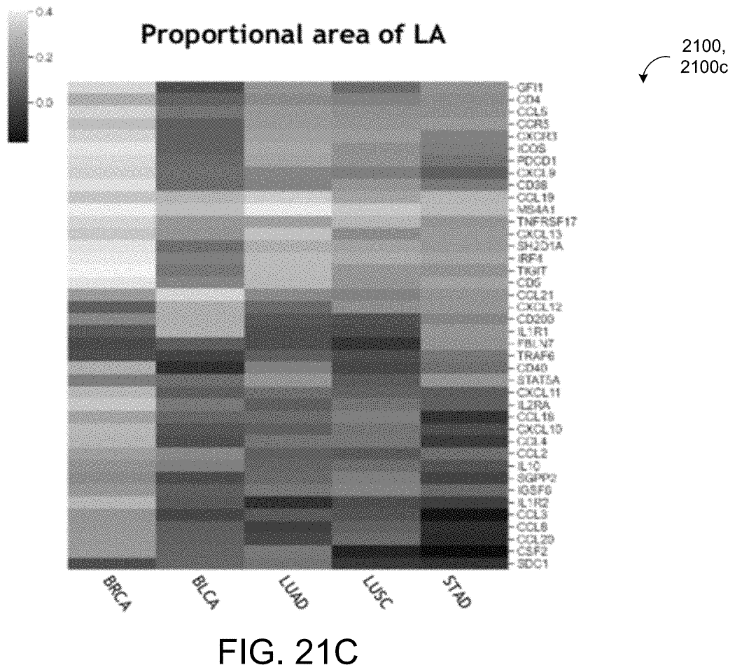

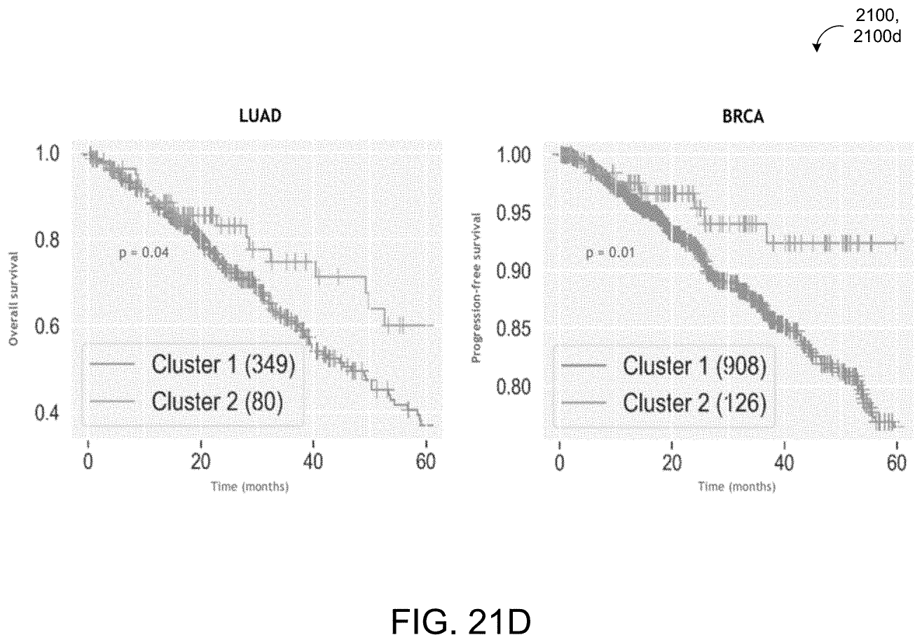

FIGS. 21A-21D illustrate graphical representations 2100 of correlation diagrams that validate the extracted TLS features 140 with gene signatures. The graphical representations 2100 correlate the TLS features 140 and gene signatures among different cancer types (x-axis) including BRCA, bladder cancer (BLCA), lung adenocarcinoma (LUAD), lung squamous cell carcinoma (LUSC), and stomach adenocarcinoma (STAD). Moreover, each graphical representation 2100 plots the TLS-induced genes along the y-axis. For instance, graphical representation 2100a (FIG. 21A) includes the TLS feature 140 of proportional area of mature TLS maturation state 312c, graphical representation 2100b (FIG. 21B) includes the TLS feature 140 for proportional area of immature TLS maturation state 312b, and graphical representation 2100c (FIG. 21C) includes the TLS feature 140 for proportional area of lymphoid aggregate TLS maturation state 312c. FIG. 2DC illustrates graphical representation 2100d of a plot depicting an x-axis as a timeline in months and a y-axis as an overall survival rate of the patients from the LUAD cancer samples and BRCA cancer samples. Thus, the graphical representation 2100d shows that the proportional area of different TLS maturations states 312 correlate with a subset of TLS-induced genes and, in particular, that the proportional area of the mature TLS maturations states 312c demonstrates prognostic value in LUAD and BRCA samples. Thus, the graphical representations 2000c shows that breast cancer samples in clusters with up-regulated chemokines have a higher long-term overall survival rate.

Anti-PD-1 antibodies that are known in the art can be used in the presently described compositions and methods. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1×10⁻⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-γ production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some embodiments, at least five, of the preceding characteristics.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

In some implementations, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; also known as toripalimab; see Si-Yang Liu et al., J. Hematol. Oncol. 10:136 (2017)), BGB-A317 (Beigene; also known as Tislelizumab; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), BCD-100 (Biocad; Kaplon et al., mAbs 10(2):183-203 (2018), and IBI308 (Innovent; see WO 2017/024465, WO 2017/025016, WO 2017/132825, and WO 2017/133540).

Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56). Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587.

Anti-PD-1 antibodies usable in the disclosed compositions and methods also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some embodiments, the anti-PD-1 antibody binds the same epitope as any of the anti-PD-1 antibodies described herein, e.g., nivolumab. The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In some implementations, the antibodies that cross-compete for binding to human PD-1 with, or bind to the same epitope region of human PD-1 antibody, nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-1 antibodies usable in the compositions and methods of the present disclosure also include antigen-binding portions of the above antibodies. It has been amply demonstrated that the antigen-binding function of an antibody can be performed by fragments of a full-length antibody.

Anti-PD-1 antibodies suitable for use in the disclosed compositions and methods are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain embodiments, the anti-PD-1 antibody or antigen-binding portion thereof cross-competes with nivolumab for binding to human PD-1.

In some examples, the anti-PD-1 antibody is administered at a dose ranging from 0.1 mg/kg to 20.0 mg/kg body weight once every 2, 3, 4, 5, 6, 7, or 8 weeks, e.g., 0.1 mg/kg to 10.0 mg/kg body weight once every 2, 3, or 4 weeks. In other embodiments, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 2 weeks. In other embodiments, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 3 weeks. In one embodiment, the anti-PD-1 antibody is administered at a dose of about 5 mg/kg body weight about once every 3 weeks. In another embodiment, the anti-PD-1 antibody, e.g., nivolumab, is administered at a dose of about 3 mg/kg body weight about once every 2 weeks. In other embodiments, the anti-PD-1 antibody, e.g., Pembrolizumab, is administered at a dose of about 2 mg/kg body weight about once every 3 weeks.

The anti-PD-1 antibody useful for the present disclosure can be administered as a flat dose. In some embodiments, the anti-PD-1 antibody is administered at a flat dose of from about 100 to about 1000 mg, from about 100 mg to about 900 mg, from about 100 mg to about 800 mg, from about 100 mg to about 700 mg, from about 100 mg to about 600 mg, from about 100 mg to about 500 mg, from about 200 mg to about 1000 mg, from about 200 mg to about 900 mg, from about 200 mg to about 800 mg, from about 200 mg to about 700 mg, from about 200 mg to about 600 mg, from about 200 mg to about 500 mg, from about 200 mg to about 480 mg, or from about 240 mg to about 480 mg, In one embodiment, the anti-PD-1 antibody is administered as a flat dose of at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg, at least about 540 mg, at least about 550 mg, at least about 560 mg, at least about 580 mg, at least about 600 mg, at least about 620 mg, at least about 640 mg, at least about 660 mg, at least about 680 mg, at least about 700 mg, or at least about 720 mg at a dosing interval of about 1, 2, 3, 4, 5, 6, 7, 8, 9, or 10 weeks. In another embodiments, the anti-PD-1 antibody is administered as a flat dose of about 200 mg to about 800 mg, about 200 mg to about 700 mg, about 200 mg to about 600 mg, about 200 mg to about 500 mg, at a dosing interval of about 1, 2, 3, or 4 weeks.

In some implementations, the anti-PD-1 antibody is administered as a flat dose of about 200 mg at about once every 3 weeks. In other embodiments, the anti-PD-1 antibody is administered as a flat dose of about 200 mg at about once every 2 weeks. In other embodiments, the anti-PD-1 antibody is administered as a flat dose of about 240 mg at about once every 2 weeks. In certain embodiments, the anti-PD-1 antibody is administered as a flat dose of about 480 mg at about once every 4 weeks.

In some additional implementations, nivolumab is administered at a flat dose of about 240 mg once about every 2 weeks. In some embodiments, nivolumab is administered at a flat dose of about 240 mg once about every 3 weeks. In some embodiments, nivolumab is administered at a flat dose of about 360 mg once about every 3 weeks. In some embodiments, nivolumab is administered at a flat dose of about 480 mg once about every 4 weeks.

Alternatively, Pembrolizumab may be administered at a flat dose of about 200 mg once about every 2 weeks. In some embodiments, Pembrolizumab is administered at a flat dose of about 200 mg once about every 3 weeks. In some embodiments, Pembrolizumab is administered at a flat dose of about 400 mg once about every 4 weeks.

In some aspects, the PD-1 inhibitor is a small molecule. In some aspects, the PD-1 inhibitor includes a millamolecule. In some aspects, the PD-1 inhibitor includes a macrocyclic peptide. The PD-1 inhibitor may include BMS-986189. In some additional aspects, the PD-1 inhibitor includes an inhibitor disclosed in International Publication No. WO2014/151634, which is incorporated by reference herein in its entirety. In some aspects, the PD-1 inhibitor includes INCMGA00012 (Incyte Corporation). In some aspects, the PD-1 inhibitor includes a combination of an anti-PD-1 antibody disclosed herein and a PD-1 small molecule inhibitor.

In some implementations, an anti-PD-L1 antibody is substituted for the anti-PD-1 antibody in any of the methods disclosed herein. Anti-PD-L1 antibodies that are known in the art can be used in the compositions and methods of the present disclosure. Examples of anti-PD-L1 antibodies useful in the compositions and methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-γ production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include monoclonal antibodies that bind specifically to human PD-L1 and exhibit at least one, in some embodiments, at least five, of the preceding characteristics.

The anti-PD-L1 antibody may be selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., JCO 36 (15suppl):TPS3113 (2018)), and CK-301 (Checkpoint Therapeutics; see Gorelik et al., AACR:Abstract 4606 (April 2016)).

Atezolizumab is a fully humanized IgG1 monoclonal anti-PD-L1 antibody. Durvalumab is a human IgG1 kappa monoclonal anti-PD-L1 antibody. Avelumab is a human IgG1 lambda monoclonal anti-PD-L1 antibody. Anti-PD-L1 antibodies usable in the disclosed compositions and methods also include isolated antibodies that bind specifically to human PD-L1 and cross-compete for binding to human PD-L1 with any anti-PD-L1 antibody disclosed herein, e.g., atezolizumab, durvalumab, and/or avelumab. In some embodiments, the anti-PD-L1 antibody binds the same epitope as any of the anti-PD-L1 antibodies described herein, e.g., atezolizumab, durvalumab, and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., atezolizumab and/or avelumab, by virtue of their binding to the same epitope region of PD-L1. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

The antibodies that cross-compete for binding to human PD-L1 with, or bind to the same epitope region of human PD-L1 antibody as, atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies usable in the compositions and methods of the disclosed disclosure also include antigen-binding portions of the above antibodies. It has been amply demonstrated that the antigen-binding function of an antibody can be performed by fragments of a full-length antibody.

Anti-PD-L1 antibodies suitable for use in the disclosed compositions and methods are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain embodiments, the anti-PD-L1 antibody or antigen-binding portion thereof cross-competes with atezolizumab, durvalumab, and/or avelumab for binding to human PD-L1.

The anti-PD-L1 antibody useful for the present disclosure can be any PD-L1 antibody that specifically binds to PD-L1, e.g., antibodies that cross-compete with durvalumab, avelumab, or atezolizumab for binding to human PD-1, e.g., an antibody that binds to the same epitope as durvalumab, avelumab, or atezolizumab. In a particular embodiment, the anti-PD-L1 antibody is durvalumab. In other embodiments, the anti-PD-L1 antibody is avelumab. In some embodiments, the anti-PD-L1 antibody is atezolizumab.

In some implementations, the anti-PD-L1 antibody is administered at a dose ranging from about 0.1 mg/kg to about 20.0 mg/kg body weight, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, about once every 2, 3, 4, 5, 6, 7, or 8 weeks.

The anti-PD-L1 antibody may be administered at a dose of about 15 mg/kg body weight at about once every 3 weeks. In other embodiments, the anti-PD-L1 antibody is administered at a dose of about 10 mg/kg body weight at about once every 2 weeks.

In some scenarios, the anti-PD-L1 antibody useful for the present disclosure is a flat dose. In some embodiments, the anti-PD-L1 antibody is administered as a flat dose of from about 200 mg to about 1600 mg, about 200 mg to about 1500 mg, about 200 mg to about 1400 mg, about 200 mg to about 1300 mg, about 200 mg to about 1200 mg, about 200 mg to about 1100 mg, about 200 mg to about 1000 mg, about 200 mg to about 900 mg, about 200 mg to about 800 mg, about 200 mg to about 700 mg, about 200 mg to about 600 mg, about 700 mg to about 1300 mg, about 800 mg to about 1200 mg, about 700 mg to about 900 mg, or about 1100 mg to about 1300 mg. In some embodiments, the anti-PD-L1 antibody is administered as a flat dose of at least about 240 mg, at least about 300 mg, at least about 320 mg, at least about 400 mg, at least about 480 mg, at least about 500 mg, at least about 560 mg, at least about 600 mg, at least about 640 mg, at least about 700 mg, at least 720 mg, at least about 800 mg, at least about 840 mg, at least about 880 mg, at least about 900 mg, at least 960 mg, at least about 1000 mg, at least about 1040 mg, at least about 1100 mg, at least about 1120 mg, at least about 1200 mg, at least about 1280 mg, at least about 1300 mg, at least about 1360 mg, or at least about 1400 mg, at a dosing interval of about 1, 2, 3, or 4 weeks. In some embodiments, the anti-PD-L1 antibody is administered as a flat dose of about 1200 mg at about once every 3 weeks. In other embodiments, the anti-PD-L1 antibody is administered as a flat dose of about 800 mg at about once every 2 weeks. In other embodiments, the anti-PD-L1 antibody is administered as a flat dose of about 840 mg at about once every 2 weeks.

Atezolizumab is administered as a flat dose of about 1200 mg once about every 3 weeks. In some examples, atezolizumab is administered as a flat dose of about 800 mg once about every 2 weeks. In other examples, atezolizumab is administered as a flat dose of about 840 mg once about every 2 weeks. Optionally, avelumab may be administered as a flat dose of about 800 mg once about every 2 weeks.

In some examples, durvalumab is administered at a dose of about 10 mg/kg once about every 2 weeks. In other examples, durvalumab is administered as a flat dose of about 800 mg/kg once about every 2 weeks. Durvalumab may optionally be administered as a flat dose of about 1200 mg/kg once about every 3 weeks.

The PD-L1 inhibitor may include a small molecule or a millamolecule. The PD-L1 inhibitor may include a macrocyclic peptide. In some implementations, the PD-L1 inhibitor includes BMS-986189. The PD-L1 inhibitor may include a millamolecule having the following formula:

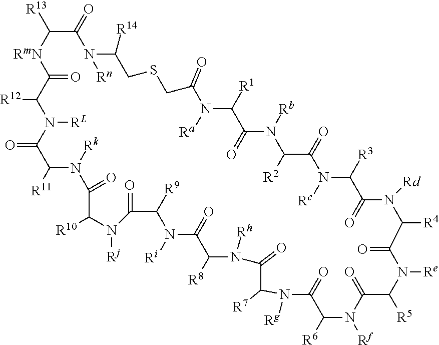

where R1-R13 are amino acid side chains, R^{a}—Rⁿ are hydrogen, methyl, or form a ring with a vicinal R group, and R14 is —C(O)NHR15, wherein R15 is hydrogen, or a glycine residue optionally substituted with additional glycine residues and/or tails which can improve pharmacokinetic properties. In some aspects, the PD-L1 inhibitor includes a compound disclosed in International Publication No. WO2014/151634, which is incorporated by reference herein in its entirety. In some aspects, the PD-L1 inhibitor includes a compound disclosed in International Publication No. WO2016/039749, WO2016/149351, WO2016/077518, WO2016/100285, WO2016/100608, WO2016/126646, WO2016/057624, WO2017/151830, WO2017/176608, WO2018/085750, WO2018/237153, or WO2019/070643, each of which is incorporated by reference herein in its entirety.

The PD-L1 inhibitor includes a small molecule PD-L1 inhibitor disclosed in International Publication No. WO2015/034820, WO2015/160641, WO2018/044963, WO2017/066227, WO2018/009505, WO2018/183171, WO2018/118848, WO2019/147662, or WO2019/169123, each of which is incorporated by reference herein in its entirety. In some implementations, the PD-L1 inhibitor includes a combination of an anti-PD-L1 antibody disclosed herein and a PD-L1 small molecule inhibitor disclosed herein.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

The non-transitory memory may be physical devices used to store programs (e.g., sequences of instructions) or data (e.g., program state information) on a temporary or permanent basis for use by a computing device. The non-transitory memory may be volatile and/or non-volatile addressable semiconductor memory. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

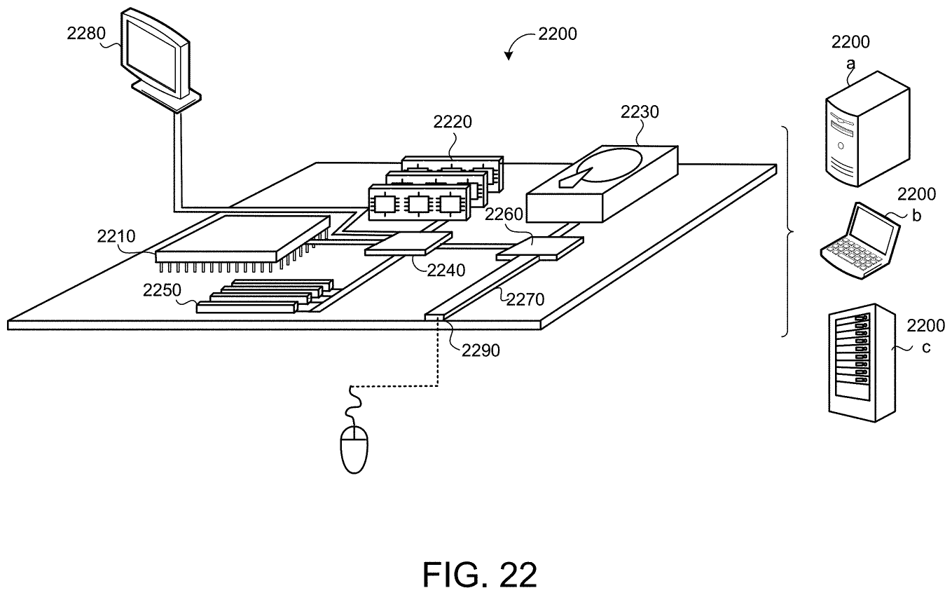

FIG. 22 is schematic view of an example computing device 2200 that may be used to implement the systems and methods described in this document. The computing device 2200 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 2200 includes a processor 2210, memory 2220, a storage device 2230, a high-speed interface/controller 2240 connecting to the memory 2220 and high-speed expansion ports 2250, and a low speed interface/controller 2260 connecting to a low speed bus 2270 and a storage device 2230. Each of the components 2210, 2220, 2230, 2240, 2250, and 2260, are interconnected using various busses, and may be mounted on a common motherboard or in other manners as appropriate. The processor 2210 can process instructions for execution within the computing device 2200, including instructions stored in the memory 2220 or on the storage device 2230 to display graphical information for a graphical user interface (GUI) on an external input/output device, such as display 2280 coupled to high speed interface 2240. In other implementations, multiple processors and/or multiple buses may be used, as appropriate, along with multiple memories and types of memory. Also, multiple computing devices 2200 may be connected, with each device providing portions of the necessary operations (e.g., as a server bank, a group of blade servers, or a multi-processor system).

The memory 2220 stores information non-transitorily within the computing device 2200. The memory 2220 may be a computer-readable medium, a volatile memory unit(s), or non-volatile memory unit(s). The non-transitory memory 2220 may be physical devices used to store programs (e.g., sequences of instructions) or data (e.g., program state information) on a temporary or permanent basis for use by the computing device 2200. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 2230 is capable of providing mass storage for the computing device 2200. In some implementations, the storage device 2230 is a computer-readable medium. In various different implementations, the storage device 2230 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 2220, the storage device 2230, or memory on processor 2210.

The high speed controller 2240 manages bandwidth-intensive operations for the computing device 2200, while the low speed controller 2260 manages lower bandwidth-intensive operations. Such allocation of duties is exemplary only. In some implementations, the high-speed controller 2240 is coupled to the memory 2220, the display 2280 (e.g., through a graphics processor or accelerator), and to the high-speed expansion ports 2250, which may accept various expansion cards (not shown). In some implementations, the low-speed controller 2260 is coupled to the storage device 2230 and a low-speed expansion port 2290. The low-speed expansion port 2290, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 2200 may be implemented in a number of different forms, as shown in the figure. For example, it may be implemented as a standard server 2200a or multiple times in a group of such servers 2200a, as a laptop computer 2200b, or as part of a rack server system 2200c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims.

What is claimed is:

1. A computer-implemented method executed on data processing hardware that causes the data processing hardware to perform operations comprising:
receiving an input histology image for a patient diagnosed with cancer, the input histology image comprising a plurality of image pixels;
processing, using a cell classification model, the input histology image to generate one or more lymphocyte density maps within the input histology image;
performing morphological image processing on the one or more lymphocyte density maps to identify one or more TLS regions within the input histology image, each TLS region represented by a respective cluster of lymphocyte cells; and
for each corresponding TLS region of the one or more TLS regions identified in the input histology image:
extracting, from the respective cluster of lymphocyte cells representing the corresponding TLS region, a respective set of TLS features; and
processing, using a TLS classification model, the respective set of TLS features to classify the corresponding TLS region as one of a first TLS maturation state, a second TLS maturation state, or a third TLS maturation state.

2. The computer-implemented method of claim 1, wherein the operations further comprise:
processing, using a tumor detection model, the input histology image to identify a tumor region within the input histology image,
wherein processing the input histology image to generate the one or more lymphocyte density maps comprises processing, using the cell classification model, the input histology image by performing single-cell imaging analysis on the tumor region identified within the input histology image to generate the one or more lymphocyte density maps.

3. The computer-implemented method of claim 2, wherein the tumor detection model is trained by:
obtaining a plurality of image tiles rasterized from a set of whole-slide histopathology images, each image tile manually annotated as including a tumor or a non-tumor; and
training, using a neural network, the tumor detection model on the plurality of image tiles to teach the tumor detection model to learn how to identify tumor regions within histology images.

4. The computer-implemented method of claim 1, wherein the cell classification model is trained by:
obtaining a plurality of image patches, each image patch comprising a corresponding plurality of human cells and manual annotations that label each human cell as a tumor cell, a lymphocyte cell, or a non-malignant cell; and
training, using a neural network, the cell classification model on the plurality of image patches to teach the cell classification model to learn how to classify individual cells in histology images as tumor cells, lymphocyte cells, or non-malignant cells.

5. The computer-implemented method of claim 1, wherein the TLS classification model is trained by:
obtaining a training dataset comprising a plurality of training histology images, each training histology image containing a tumor microenvironment and comprising manual annotations that identify:
one or more TLS regions in the training histology image, each TLS region represented by a respective cluster of lymphocyte cells; and
for each corresponding TLS region, a ground-truth TLS maturation state indicating that the corresponding TLS region comprises a first TLS maturation state, a second TLS maturation state, or a third TLS maturation state;
for each TLS region, extracting, from the respective cluster of lymphocyte cells representing the TLS region, a respective set of training TLS features; and
training the TLS classification model on the respective set of training TLS features extracted for each TLS region to teach the TLS classification model to learn how to predict the ground-truth TLS grade for each corresponding TLS region.

6. The computer-implemented method of claim 5, wherein training the TLS classification model comprises training the TLS classification model using a classification and regression trees (CART) algorithm.

7. The computer-implemented method of claim 1, wherein:
the first TLS maturation state comprises a dense aggregate of at least a threshold number of lymphocytes that do not contain high endothelial venules or germinal centers;
the second TLS maturation state comprises an immature TLS comprising a dense aggregate of at least the threshold number of lymphocytes that contain high endothelial venules and do not contain any germinal centers; and
the third TLS maturation state comprises a mature TLS comprising a dense aggregate of at least the threshold number of lymphocytes that contain high endothelial venules and germinal centers.

8. The computer-implemented method of claim 1, wherein the operations further comprise:
for each corresponding TLS region of the one or more TLS regions identified in the input histology image, generating a respective pixel mask that highlights at least a perimeter of the corresponding TLS region;
generating an output image that augments the input histology image by overlaying the respective pixel mask generated for each of the TLS regions onto the input histology image; and
providing, for display on a screen in communication with the data processing hardware, the output image.

9. The computer-implemented method of claim 8, wherein:
the respective pixel mask generated for each corresponding TLS region classified as the first maturation state comprises a first pixel mask;
the respective pixel mask generated for each corresponding TLS region classified as the second maturation state comprises a second pixel mask that is visually distinguishable from the second pixel mask; and
the respective pixel mask generated for each corresponding TLS region classified as the third maturation state comprises a third pixel mask that is visually distinguishable from the first pixel mask and the second pixel mask.

10. The computer-implemented method of claim 1, wherein the respective set of TLS features extracted from the respective cluster of lymphocyte cells comprises an area of the corresponding TLS region, a roundness of the corresponding TLS region, and a skewness of the corresponding TLS region.

11. The computer-implemented method of claim 1, wherein the operations further comprise determining an overall TLS score for the input histology image based on the TLS maturation states for the one or more TLS regions identified in the histology image and the TLS features extracted from the one or more TLS regions identified in the histology image.

12. The computer-implemented method of claim 11, wherein the operations further comprise determining a treatment recommendation to treat the patient using immunotherapy based on the overall TLS score.

13. The computer-implemented method of claim 12, wherein the immunotherapy comprises at least one of a PD-1 inhibitor or a PD-L1 inhibitor.

14. The computer-implemented method of claim 11, wherein the operations further comprise determining a predictive score of the patient's response to immunotherapy based on the TLS maturation states for the one or more TLS regions identified in the histology image and the TLS features extracted from the one or more TLS regions identified in the histology image.

15. A system comprising:
data processing hardware; and
memory hardware in communication with the data processing hardware, the memory hardware storing instructions that when executed on the data processing hardware cause the data processing hardware to perform operations comprising:
receiving an input histology image for a patient diagnosed with cancer, the input histology image comprising a plurality of image pixels;
processing, using a cell classification model, the input histology image to generate one or more lymphocyte density maps within the input histology image;
performing morphological image processing on the one or more lymphocyte density maps to identify one or more TLS regions within the input histology image, each TLS region represented by a respective cluster of lymphocyte cells; and
for each corresponding TLS region of the one or more TLS regions identified in the input histology image:
extracting, from the respective cluster of lymphocyte cells representing the corresponding TLS region, a respective set of TLS features; and
processing, using a TLS classification model, the respective set of TLS features to classify the corresponding TLS region as one of a first TLS maturation state, a second TLS maturation state, or a third TLS maturation state.

16. The system of claim 15, wherein the operations further comprise:
processing, using a tumor detection model, the input histology image to identify a tumor region within the input histology image,
wherein processing the input histology image to generate the one or more lymphocyte density maps comprises processing, using the cell classification model, the input histology image by performing single-cell imaging analysis on the tumor region identified within the input histology image to generate the one or more lymphocyte density maps.

17. The system of claim 16, wherein the tumor detection model is trained by:
obtaining a plurality of image tiles rasterized from a set of whole-slide histopathology images, each image tile manually annotated as including a tumor or a non-tumor; and
training, using a neural network, the tumor detection model on the plurality of image tiles to teach the tumor detection model to learn how to identify tumor regions within histology images.

18. The system of claim 15, wherein the cell classification model is trained by:
obtaining a plurality of image patches, each image patch comprising a corresponding plurality of human cells and manual annotations that label each human cell as a tumor cell, a lymphocyte cell, or a non-malignant cell; and
training, using a neural network, the cell classification model on the plurality of image patches to teach the cell classification model to learn how to classify individual cells in histology images as tumor cells, lymphocyte cells, or non-malignant cells.

19. The system of claim 15, wherein the TLS classification model is trained by:
obtaining a training dataset comprising a plurality of training histology images, each training histology image containing a tumor microenvironment and comprising manual annotations that identify:
one or more TLS regions in the training histology image, each TLS region represented by a respective cluster of lymphocyte cells; and
for each corresponding TLS region, a ground-truth TLS maturation state indicating that the corresponding TLS region comprises a first TLS maturation state, a second TLS maturation state, or a third TLS maturation state;
for each TLS region, extracting, from the respective cluster of lymphocyte cells representing the TLS region, a respective set of training TLS features; and
training the TLS classification model on the respective set of training TLS features extracted for each TLS region to teach the TLS classification model to learn how to predict the ground-truth TLS grade for each corresponding TLS region.

20. The system of claim 19, wherein training the TLS classification model comprises training the TLS classification model using a classification and regression trees (CART) algorithm.

21. The system of claim 15, wherein:
the first TLS maturation state comprises a dense aggregate of at least a threshold number of lymphocytes that do not contain high endothelial venules or germinal centers;
the second TLS maturation state comprises an immature TLS comprising a dense aggregate of at least the threshold number of lymphocytes that contain high endothelial venules and do not contain any germinal centers; and
the third TLS maturation state comprises a mature TLS comprising a dense aggregate of at least the threshold number of lymphocytes that contain high endothelial venules and germinal centers.

22. The system of claim 15, wherein the operations further comprise:
for each corresponding TLS region of the one or more TLS regions identified in the input histology image, generating a respective pixel mask that highlights at least a perimeter of the corresponding TLS region;
generating an output image that augments the input histology image by overlaying the respective pixel mask generated for each of the TLS regions onto the input histology image; and
providing, for display on a screen in communication with the data processing hardware, the output image.

23. The system of claim 22, wherein:
the respective pixel mask generated for each corresponding TLS region classified as the first maturation state comprises a first pixel mask;
the respective pixel mask generated for each corresponding TLS region classified as the second maturation state comprises a second pixel mask that is visually distinguishable from the second pixel mask; and
the respective pixel mask generated for each corresponding TLS region classified as the third maturation state comprises a third pixel mask that is visually distinguishable from the first pixel mask and the second pixel mask.

24. The system of claim 15, wherein the respective set of TLS features extracted from the respective cluster of lymphocyte cells comprises an area of the corresponding TLS region, a roundness of the corresponding TLS region, and a skewness of the corresponding TLS region.

25. The system of claim 15, wherein the operations further comprise determining an overall TLS score for the input histology image based on the TLS maturation states for the one or more TLS regions identified in the histology image and the TLS features extracted from the one or more TLS regions identified in the histology image.

26. The system of claim 25, wherein the operations further comprise determining a treatment recommendation to treat the patient using immunotherapy based on the overall TLS score.

27. The system of claim 26, wherein the immunotherapy comprises at least one of a PD-1 inhibitor or a PD-L1 inhibitor.

28. The system of claim 25, wherein the operations further comprise determining a predictive score of the patient's response to immunotherapy based on the TLS maturation states for the one or more TLS regions identified in the histology image and the TLS features extracted from the one or more TLS regions identified in the histology image.
